SEQUENCE LISTING (16 sequences)

SEQ ID NO: 1 - DNA, 10365 bp, Homo Sapiens
  misc_feature (9804)..(10365): n=a,t,g or c
gtgctgtgctgccagttgtcatctttgaacaaactgatgcagtgctggtttaactcttcctctttttggagtaagaaactttggaggcctgtgtccttctagaagtttgctgagcaaatgttaatgaccagccacgggcgtccctgctgtgagctctggccgctgccttccagggctcccgagccacacgctgggcgtgctggctgagggaacatggcttgttggcctcagctgaggttgctgctgtggaagaacctcactttcagaagaagacaaacatgtcagctgttactggaagtggcctggcctctatttatcttcctgatcctgatctctgttcggctgagctacccaccctatgaacaacatgaatgccattttccaaataaagccatgccctctgcaggaacacttccttgggttcaggggattatctgtaatgccaacaacccctgtttccgttacccgactcctggggaggctcccggagttgttggaaactttaacaaatccattgtggctcgcctgttctcagatgctcggaggcttcttttatacagccagaaagacaccagcatgaaggacatgcgcaaagttctgagaacattacagcagatcaagaaatccagctcaaacttgaagcttcaagatttcctggtggacaatgaaaccttctctgggttcctatatcacaacctctctctcccaaagtctactgtggacaagatgctgagggctgatgtcattctccacaaggtatttttgcaaggctaccagttacatttgacaagtctgtgcaatggatcaaaatcagaagagatgattcaacttggtgaccaagaagtttctgagctttgtggcctaccaaaggagaaactggctgcagcagagcgagtacttcgttccaacatggacatcctgaagccaatcctgagaacactaaactctacatctcccttcccgagcaaggagctggctgaagccacaaaaacattgctgcatagtcttgggactctggcccaggagctgttcagcatgagaagctggagtgacatgcgacaggaggtgatgtttctgaccaatgtgaacagctccagctcctccacccaaatctaccaggctgtgtctcgtattgtctgcgggcatcccgagggaggggggctgaagatcaagtctctcaactggtatgaggacaacaactacaaagccctctttggaggcaatggcactgaggaagatgctgaaaccttctatgacaactctacaactccttactgcaatgatttgatgaagaatttggagtctagtcctctttcccgcattatctggaaagctctgaagccgctgctcgttgggaagatcctgtatacacctgacactccagccacaaggcaggtcatggctgaggtgaacaagaccttccaggaactggctgtgttccatgatctggaaggcatgtgggaggaactcagccccaagatctggaccttcatggagaacagccaagaaatggaccttgtccggatgctgttggacagcagggacaatgaccacttttgggaacagcagttggatggcttagattggacagcccaagacatcgtggcgtttttggccaagcacccagaggatgtccagtccagtaatggttctgtgtacacctggagagaagctttcaacgagactaaccaggcaatccggaccatatctcgcttcatggagtgtgtcaacctgaacaagctagaacccatagcaacagaagtctggctcatcaacaagtccatggagctgctggatgagaggaagttctgggctggtattgtgttcactggaattactccaggcagcattgagctgccccatcatgtcaagtacaagatccgaatggacattgacaatgtggagaggacaaataaaatcaaggatgggtactgggaccctggtcctcgagctgacccctttgaggacatgcggtacgtctgggggggcttcgcctacttgcaggatgtggtggagcaggcaatcatcagggtgctgacgggcaccgagaagaaaactggtgtctatatgcaacagatgccctatccctgttacgttgatgacatctttctgcgggtgatgagccggtcaatgcccctcttcatgacgctggcctggatttactcagtggctgtgatcatcaagggcatcgtgtatgagaaggaggcacggctgaaagagaccatgcggatcatgggcctggacaacagcatactctggtttagctggttcattagtagcctcattcctcttcttgtgagcgctggcctgctagtggtcatcctgaagttaggaaacctgctgccctacagtgatcccagcgtggtgtttgtcttcctgtccgtgtttgctgtggtgacaatcctgcagtgcttcctgattagcacactcttctccagagccaacctggcagcagcctgtgggggcatcatctacttcacgctgtacctgccctacgtcctgtgtgtggcatggcaggactacgtgggcttcacactcaagatcttcgctagcctgctgtctcctgtggcttttgggtttggctgtgagtactttgccctttttgaggagcagggcattggagtgcagtgggacaacctgtttgagagtcctgtggaggaagatggcttcaatctcaccacttcgatctccatgatgctgtttgacaccttcctctatggggtgatgacctggtacattgaggctgtctttccaggccagtacggaattcccaggccctggtattttccttgcaccaagtcctactggtttggcgaggaaagtgatgagaagagccaccctggttccaaccagaagagaatgtcagaaatctgcatggaggaggaacccacccacttgaagctgggcgtgtccattcagaacctggtaaaagtctaccgagatgggatgaaggtggctgtcgatggcctggcactgaatttttatgagggccagatcacctccttcctgggccacaatggagcggggaagacgaccaccatgtcaatcctgaccgggttgttccccccgacctcgggcaccgcctacatcctgggaaaagacattcgctctgagatgagcaccatccggcagaacctgggggtctgtccccagcataacgtgctgtttgacatgctgactgtcgaagaacacatctggttctatgcccgcttgaaagggctctctgagaagcacgtgaaggcggagatggagcagatggccctggatgttggtttgccatcaagcaagctgaaaagcaaaacaagccagctgtcaggtggaatgcagagaaagctatctgtggccttggcctttgtcgggggatctaaggttgtcattctggatgaacccacagctggtgtggacccttactcccgcaggggaatatgggagctgctgctgaaataccgacaaggccgcaccattattctctctacacaccacatggatgaagcggacgtcctgggggacaggattgccatcatctcccatgggaagctgtgctgtgtgggctcctccctgtttctgaagaaccagctgggaacaggctactacctgaccttggtcaagaaagatgtggaatcctccctcagttcctgcagaaacagtagtagcactgtgtcatacctgaaaaaggaggacagtgtttctcagagcagttctgatgctggcctgggcagcgaccatgagagtgacacgctgaccatcgatgtctctgctatctccaacctcatcaggaagcatgtgtctgaagcccggctggtggaagacatagggcatgagctgacctatgtgctgccatatgaagctgctaaggagggagcctttgtggaactctttcatgagattgatgaccggctctcagacctgggcatttctagttatggcatctcagagacgaccctggaagaaatattcctcaaggtggccgaagagagtggggtggatgctgagacctcagatggtaccttgccagcaagacgaaacaggcgggccttcggggacaagcagagctgtcttcgcccgttcactgaagatgatgctgctgatccaaatgattctgacatagacccagaatccagagagacagacttgctcagtgggatggatggcaaagggtcctaccaggtgaaaggctggaaacttacacagcaacagtttgtggcccttttgtggaagagactgctaattgccagacggagtcggaaaggattttttgctcagattgtcttgccagctgtgtttgtctgcattgcccttgtgttcagcctgatcgtgccaccctttggcaagtaccccagcctggaacttcagccctggatgtacaacgaacagtacacatttgtcagcaatgatgctcctgaggacacgggaaccctggaactcttaaacgccctcaccaaagaccctggcttcgggacccgctgtatggaaggaaacccaatcccagacacgccctgccaggcaggggaggaagagtggaccactgccccagttccccagaccatcatggacctcttccagaatgggaactggacaatgcagaacccttcacctgcatgccagtgtagcagcgacaaaatcaagaagatgctgcctgtgtgtcccccaggggcaggggggctgcctcctccacaaagaaaacaaaacactgcagatatccttcaggacctgacaggaagaaacatttcggattatctggtgaagacgtatgtgcagatcatagccaaaagcttaaagaacaagatctgggtgaatgagtttaggtatggcggcttttccctgggtgtcagtaatactcaagcacttcctccgagtcaagaagttaatgatgccatcaaacaaatgaagaaacacctaaagctggccaaggacagttctgcagatcgatttctcaacagcttgggaagatttatgacaggactggacaccagaaataatgtcaaggtgtggttcaataacaagggctggcatgcaatcagctctttcctgaatgtcatcaacaatgccattctccgggccaacctgcaaaagggagagaaccctagccattatggaattactgctttcaatcatcccctgaatctcaccaagcagcagctctcagaggtggctctgatgaccacatcagtggatgtccttgtgtccatctgtgtcatctttgcaatgtccttcgtcccagccagctttgtcgtattcctgatccaggagcgggtcagcaaagcaaaacacctgcagttcatcagtggagtgaagcctgtcatctactggctctctaattttgtctgggatatgtgcaattacgttgtccctgccacactggtcattatcatcttcatctgcttccagcagaagtcctatgtgtcctccaccaatctgcctgtgctagcccttctacttttgctgtatgggtggtcaatcacacctctcatgtacccagcctcctttgtgttcaagatccccagcacagcctatgtggtgctcaccagcgtgaacctcttcattggcattaatggcagcgtggccacctttgtgctggagctgttcaccgacaataagctgaataatatcaatgatatcctgaagtccgtgttcttgatcttcccacatttttgcctgggacgagggctcatcgacatggtgaaaaaccaggcaatggctgatgccctggaaaggtttggggagaatcgctttgtgtcaccattatcttgggacttggtgggacgaaacctcttcgccatggccgtggaaggggtggtgttcttcctcattactgttctgatccagtacagattcttcatcaggcccagacctgtaaatgcaaagctatctcctctgaatgatgaagatgaagatgtgaggcgggaaagacagagaattcttgatggtggaggccagaatgacatcttagaaatcaaggagttgacgaagatatatagaaggaagcggaagcctgctgttgacaggatttgcgtgggcattcctcctggtgagtgctttgggctcctgggagttaatggggctggaaaatcatcaactttcaagatgttaacaggagataccactgttaccagaggagatgctttccttaacaaaaatagtatcttatcaaacatccatgaagtacatcagaacatgggctactgccctcagtttgatgccatcacagagctgttgactgggagagaacacgtggagttctttgcccttttgagaggagtcccagagaaagaagttggcaaggttggtgagtgggcgattcggaaactgggcctcgtgaagtatggagaaaaatatgctggtaactatagtggaggcaacaaacgcaagctctctacagccatggctttgatcggcgggcctcctgtggtgtttctggatgaacccaccacaggcatggatcccaaagcccggcggttcttgtggaattgtgccctaagtgttgtcaaggaggggagatcagtagtgcttacatctcatagtatggaagaatgtgaagctctttgcactaggatggcaatcatggtcaatggaaggttcaggtgccttggcagtgtccagcatctaaaaaataggtttggagatggttatacaatagttgtacgaatagcagggtccaacccggacctgaagcctgtccaggatttctttggacttgcatttcctggaagtgttctaaaagagaaacaccggaacatgctacaataccagcttccatcttcattatcttctctggccaggatattcagcatcctctcccagagcaaaaagcgactccacatagaagactactctgtttctcagacaacacttgaccaagtatttgtgaactttgccaaggaccaaagtgatgatgaccacttaaaagacctctcattacacaaaaaccagacagtagtggacgttgcagttctcacatcttttctacaggatgagaaagtgaaagaaagctatgtatgaagaatcctgttcatacggggtggctgaaagtaaagaggaactagactttcctttgcaccatgtgaagtgttgtggagaaaagagccagaagttgatgtgggaagaagtaaactggatactgtactgatactattcaatgcaatgcaattcaatgcaatgaaaacaaaattccattacaggggcagtgcctttgtagcctatgtcttgtatggctctcaagtgaaagacttgaatttagttttttacctatacctatgtgaaactctattatggaacccaatggacatatgggtttgaactcacactttttttttttttttgttcctgtgtattctcattggggttgcaacaataattcatcaagtaatcatggccagcgattattgatcaaaatcaaaaggtaatgcacatcctcattcactaagccatgccatgcccaggagactggtttcccggtgacacatccattgctggcaatgagtgtgccagagttattagtgccaagtttttcagaaagtttgaagcaccatggtgtgtcatgctcacttttgtgaaagctgctctgctcagagtctatcaacattgaatatcagttgacagaatggtgccatgcgtggctaacatcctgctttgattccctctgataagctgttctggtggcagtaacatgcaacaaaaatgtgggtgtctctaggcacgggaaacttggttccattgttatattgtcctatgcttcgagccatgggtctacagggtcatccttatgagactcttaaatatacttagatcctggtaagaggcaaagaatcaacagccaaactgctggggctgcaagctgctgaagccagggcatgggattaaagagattgtgcgttcaaacctagggaagcctgtgcccatttgtcctgactgtctgctaacatggtacactgcatctcaagatgtttatctgacacaagtgtattatttctggctttttgaattaatctagaaaatgaaaagatggagttgtattttgacaaaaatgtttgtactttttaatgttatttggaattttaagttctatcagtgacttctgaatccttagaatggcctctttgtagaaccctgtggtatagaggagtatggccactgccccactatttttattttcttatgtaagtttgcatatcagtcatgactagtgcctagaaagcaatgtgatggtcaggatctcatgacattatatttgagtttctttcagatcatttaggatactcttaatctcacttcatcaatcaaatattttttgagtgtatgctgtagctgaaagagtatgtacgtacgtataagactagagagatattaagtctcagtacacttcctgtgccatgttattcagctcactggtttacaaatataggttgtcttgtggttgtaggagcccactgtaacaatattgggcagccttttttttttttttttaattgcaacaatgcaaaagccaagaaagtataagggtcacaagtttaaacaatgaattcttcaacagggaaaacagctagcttgaaaacttgctgaaaaacacaacttgtgtttatggcatttagtaccttcaaataattggctttgcagatattggataccccattaaatctgacagtctcaaatttttcatctcttcaatcactagtcaagaaaaatataaaaacaacaaatacttccatatggagcatttttcagagttttctaacccagtcttatttttctagtcagtaaacatttgtaaaaatactgtttcactaatacttactgttaactgtcttgagagaaaagaaaaatatgagagaactattgtttggggaagttcaagtgatctttcaatatcattactaacttcttccactttttccaaaatttgaatattaacgctaaaggtgtaagacttcagatttcaaattaatctttctatattttttaaatttacagaatattatataacccactgctgaaaaagaaaaaaatgattgttttagaagttaaagtcaatattgattttaaatataagtaatgaaggcatatttccaataactagtgatatggcatcgttgcaatttacagtatcttcaaaaatacagaatttatagaataatttctcctcatttaatatttttcaaaatcaaagttatggtttcctcattttactaaaatcgtattctaattcttcattatagtaaatctatgagcaactccttacttcggttcctctgatttcaaggccatattttaaaaaatcaaaaggcactgtgaactattttgaagaaaacacgacattttaatacagattgaaaggacctcttctgaagctagaaacaatctatagttatacatcttcattaatactgtgttaccttttaaaatagtaattttttacattttcctgtgtaaacctaattgtggtagaaatttttaccaactctatactcaatcaagcaaaatttctgtatattccctgtggaatgtacctatgtgagtttcagaaattctcaaaatacgtgttcaaaaatttctgcttttgcatctttgggacacctcagaaaacttattaacaactgtgaatatgagaaatacagaagaaaataataagccctctatacataaatgcccagcacaattcattgttaaaaaacaaccaaacctcacactactgtatttcattatctgtactgaaagcaaatgctttgtgactattaaatgttgcacatcattcattcactgtatagtaatcattgactaaagccatttgctgtgttttcttcttgtggntgnatatatcaggtaaaatattttccaaagagccatgtgtcatgtaatactgaaccctttgatattgagacattaatttggacccttggtattatctactagaataatgtaatactgnagaaatattgctctaattctttcaaaatggtgcatcccccttaaaangttctatttccataaggatttagcttgcttatcccttcttataccctaagatgaagctgtttttgtgctctttgttcatcattggccctcattccaagcactttacgctgtctgtaatgggatctatttttgcactggaatatctgagaattgcaaaactagacaaaagtttcacaacagatttctaagttaaatcattttcattaaaaggaaaaaagaaaaaaaattttgtatgtcaataactttatatgaagtattaaaatgcatatttctatgttgtaatataatgagtcacaaaataaagctgtgacagttctgttaaaaaaaaaaaaaaaaaaaaaaaaaaaaaaaaaaaaaaaaaaaaaaaaaaaaaaaaaaaaaaaaaaaaaaaaaaaa

SEQ ID NO: 2 - DNA, 10381 bp, Homo sapiens
  misc_feature (9820)..(10381): n=a,t,g or c
tggaatgaagaaagataaatggggagaagggaactcacattgctttggcacttaaattaagccatgtactgtgttgggaaattatttatattatctcgttgaatccacagtagaacacagttgaacaccatacaagttaatgaccagccacgggcgtccctgctgtgagctctggccgctgccttccagggctcccgagccacacgctgggcgtgctggctgagggaacatggcttgttggcctcagctgaggttgctgctgtggaagaacctcactttcagaagaagacaaacatgtcagctgttactggaagtggcctggcctctatttatcttcctgatcctgatctctgttcggctgagctacccaccctatgaacaacatgaatgccattttccaaataaagccatgccctctgcaggaacacttccttgggttcaggggattatctgtaatgccaacaacccctgtttccgttacccgactcctggggaggctcccggagttgttggaaactttaacaaatccattgtggctcgcctgttctcagatgctcggaggcttcttttatacagccagaaagacaccagcatgaaggacatgcgcaaagttctgagaacattacagcagatcaagaaatccagctcaaacttgaagcttcaagatttcctggtggacaatgaaaccttctctgggttcctatatcacaacctctctctcccaaagtctactgtggacaagatgctgagggctgatgtcattctccacaaggtatttttgcaaggctaccagttacatttgacaagtctgtgcaatggatcaaaatcagaagagatgattcaacttggtgaccaagaagtttctgagctttgtggcctaccaaaggagaaactggctgcagcagagcgagtacttcgttccaacatggacatcctgaagccaatcctgagaacactaaactctacatctcccttcccgagcaaggagctggctgaagccacaaaaacattgctgcatagtcttgggactctggcccaggagctgttcagcatgagaagctggagtgacatgcgacaggaggtgatgtttctgaccaatgtgaacagctccagctcctccacccaaatctaccaggctgtgtctcgtattgtctgcgggcatcccgagggaggggggctgaagatcaagtctctcaactggtatgaggacaacaactacaaagccctctttggaggcaatggcactgaggaagatgctgaaaccttctatgacaactctacaactccttactgcaatgatttgatgaagaatttggagtctagtcctctttcccgcattatctggaaagctctgaagccgctgctcgttgggaagatcctgtatacacctgacactccagccacaaggcaggtcatggctgaggtgaacaagaccttccaggaactggctgtgttccatgatctggaaggcatgtgggaggaactcagccccaagatctggaccttcatggagaacagccaagaaatggaccttgtccggatgctgttggacagcagggacaatgaccacttttgggaacagcagttggatggcttagattggacagcccaagacatcgtggcgtttttggccaagcacccagaggatgtccagtccagtaatggttctgtgtacacctggagagaagctttcaacgagactaaccaggcaatccggaccatatctcgcttcatggagtgtgtcaacctgaacaagctagaacccatagcaacagaagtctggctcatcaacaagtccatggagctgctggatgagaggaagttctgggctggtattgtgttcactggaattactccaggcagcattgagctgccccatcatgtcaagtacaagatccgaatggacattgacaatgtggagaggacaaataaaatcaaggatgggtactgggaccctggtcctcgagctgacccctttgaggacatgcggtacgtctgggggggcttcgcctacttgcaggatgtggtggagcaggcaatcatcagggtgctgacgggcaccgagaagaaaactggtgtctatatgcaacagatgccctatccctgttacgttgatgacatctttctgcgggtgatgagccggtcaatgcccctcttcatgacgctggcctggatttactcagtggctgtgatcatcaagggcatcgtgtatgagaaggaggcacggctgaaagagaccatgcggatcatgggcctggacaacagcatactctggtttagctggttcattagtagcctcattcctcttcttgtgagcgctggcctgctagtggtcatcctgaagttaggaaacctgctgccctacagtgatcccagcgtggtgtttgtcttcctgtccgtgtttgctgtggtgacaatcctgcagtgcttcctgattagcacactcttctccagagccaacctggcagcagcctgtgggggcatcatctacttcacgctgtacctgccctacgtcctgtgtgtggcatggcaggactacgtgggcttcacactcaagatcttcgctagcctgctgtctcctgtggcttttgggtttggctgtgagtactttgccctttttgaggagcagggcattggagtgcagtgggacaacctgtttgagagtcctgtggaggaagatggcttcaatctcaccacttcgatctccatgatgctgtttgacaccttcctctatggggtgatgacctggtacattgaggctgtctttccaggccagtacggaattcccaggccctggtattttccttgcaccaagtcctactggtttggcgaggaaagtgatgagaagagccaccctggttccaaccagaagagaatgtcagaaatctgcatggaggaggaacccacccacttgaagctgggcgtgtccattcagaacctggtaaaagtctaccgagatgggatgaaggtggctgtcgatggcctggcactgaatttttatgagggccagatcacctccttcctgggccacaatggagcggggaagacgaccaccatgtcaatcctgaccgggttgttccccccgacctcgggcaccgcctacatcctgggaaaagacattcgctctgagatgagcaccatccggcagaacctgggggtctgtccccagcataacgtgctgtttgacatgctgactgtcgaagaacacatctggttctatgcccgcttgaaagggctctctgagaagcacgtgaaggcggagatggagcagatggccctggatgttggtttgccatcaagcaagctgaaaagcaaaacaagccagctgtcaggtggaatgcagagaaagctatctgtggccttggcctttgtcgggggatctaaggttgtcattctggatgaacccacagctggtgtggacccttactcccgcaggggaatatgggagctgctgctgaaataccgacaaggccgcaccattattctctctacacaccacatggatgaagcggacgtcctgggggacaggattgccatcatctcccatgggaagctgtgctgtgtgggctcctccctgtttctgaagaaccagctgggaacaggctactacctgaccttggtcaagaaagatgtggaatcctccctcagttcctgcagaaacagtagtagcactgtgtcatacctgaaaaaggaggacagtgtttctcagagcagttctgatgctggcctgggcagcgaccatgagagtgacacgctgaccatcgatgtctctgctatctccaacctcatcaggaagcatgtgtctgaagcccggctggtggaagacatagggcatgagctgacctatgtgctgccatatgaagctgctaaggagggagcctttgtggaactctttcatgagattgatgaccggctctcagacctgggcatttctagttatggcatctcagagacgaccctggaagaaatattcctcaaggtggccgaagagagtggggtggatgctgagacctcagatggtaccttgccagcaagacgaaacaggcgggccttcggggacaagcagagctgtcttcgcccgttcactgaagatgatgctgctgatccaaatgattctgacatagacccagaatccagagagacagacttgctcagtgggatggatggcaaagggtcctaccaggtgaaaggctggaaacttacacagcaacagtttgtggcccttttgtggaagagactgctaattgccagacggagtcggaaaggattttttgctcagattgtcttgccagctgtgtttgtctgcattgcccttgtgttcagcctgatcgtgccaccctttggcaagtaccccagcctggaacttcagccctggatgtacaacgaacagtacacatttgtcagcaatgatgctcctgaggacacgggaaccctggaactcttaaacgccctcaccaaagaccctggcttcgggacccgctgtatggaaggaaacccaatcccagacacgccctgccaggcaggggaggaagagtggaccactgccccagttccccagaccatcatggacctcttccagaatgggaactggacaatgcagaacccttcacctgcatgccagtgtagcagcgacaaaatcaagaagatgctgcctgtgtgtcccccaggggcaggggggctgcctcctccacaaagaaaacaaaacactgcagatatccttcaggacctgacaggaagaaacatttcggattatctggtgaagacgtatgtgcagatcatagccaaaagcttaaagaacaagatctgggtgaatgagtttaggtatggcggcttttccctgggtgtcagtaatactcaagcacttcctccgagtcaagaagttaatgatgccatcaaacaaatgaagaaacacctaaagctggccaaggacagttctgcagatcgatttctcaacagcttgggaagatttatgacaggactggacaccagaaataatgtcaaggtgtggttcaataacaagggctggcatgcaatcagctctttcctgaatgtcatcaacaatgccattctccgggccaacctgcaaaagggagagaaccctagccattatggaattactgctttcaatcatcccctgaatctcaccaagcagcagctctcagaggtggctctgatgaccacatcagtggatgtccttgtgtccatctgtgtcatctttgcaatgtccttcgtcccagccagctttgtcgtattcctgatccaggagcgggtcagcaaagcaaaacacctgcagttcatcagtggagtgaagcctgtcatctactggctctctaattttgtctgggatatgtgcaattacgttgtccctgccacactggtcattatcatcttcatctgcttccagcagaagtcctatgtgtcctccaccaatctgcctgtgctagcccttctacttttgctgtatgggtggtcaatcacacctctcatgtacccagcctcctttgtgttcaagatccccagcacagcctatgtggtgctcaccagcgtgaacctcttcattggcattaatggcagcgtggccacctttgtgctggagctgttcaccgacaataagctgaataatatcaatgatatcctgaagtccgtgttcttgatcttcccacatttttgcctgggacgagggctcatcgacatggtgaaaaaccaggcaatggctgatgccctggaaaggtttggggagaatcgctttgtgtcaccattatcttgggacttggtgggacgaaacctcttcgccatggccgtggaaggggtggtgttcttcctcattactgttctgatccagtacagattcttcatcaggcccagacctgtaaatgcaaagctatctcctctgaatgatgaagatgaagatgtgaggcgggaaagacagagaattcttgatggtggaggccagaatgacatcttagaaatcaaggagttgacgaagatatatagaaggaagcggaagcctgctgttgacaggatttgcgtgggcattcctcctggtgagtgctttgggctcctgggagttaatggggctggaaaatcatcaactttcaagatgttaacaggagataccactgttaccagaggagatgctttccttaacaaaaatagtatcttatcaaacatccatgaagtacatcagaacatgggctactgccctcagtttgatgccatcacagagctgttgactgggagagaacacgtggagttctttgcccttttgagaggagtcccagagaaagaagttggcaaggttggtgagtgggcgattcggaaactgggcctcgtgaagtatggagaaaaatatgctggtaactatagtggaggcaacaaacgcaagctctctacagccatggctttgatcggcgggcctcctgtggtgtttctggatgaacccaccacaggcatggatcccaaagcccggcggttcttgtggaattgtgccctaagtgttgtcaaggaggggagatcagtagtgcttacatctcatagtatggaagaatgtgaagctctttgcactaggatggcaatcatggtcaatggaaggttcaggtgccttggcagtgtccagcatctaaaaaataggtttggagatggttatacaatagttgtacgaatagcagggtccaacccggacctgaagcctgtccaggatttctttggacttgcatttcctggaagtgttctaaaagagaaacaccggaacatgctacaataccagcttccatcttcattatcttctctggccaggatattcagcatcctctcccagagcaaaaagcgactccacatagaagactactctgtttctcagacaacacttgaccaagtatttgtgaactttgccaaggaccaaagtgatgatgaccacttaaaagacctctcattacacaaaaaccagacagtagtggacgttgcagttctcacatcttttctacaggatgagaaagtgaaagaaagctatgtatgaagaatcctgttcatacggggtggctgaaagtaaagaggaactagactttcctttgcaccatgtgaagtgttgtggagaaaagagccagaagttgatgtgggaagaagtaaactggatactgtactgatactattcaatgcaatgcaattcaatgcaatgaaaacaaaattccattacaggggcagtgcctttgtagcctatgtcttgtatggctctcaagtgaaagacttgaatttagttttttacctatacctatgtgaaactctattatggaacccaatggacatatgggtttgaactcacactttttttttttttttgttcctgtgtattctcattggggttgcaacaataattcatcaagtaatcatggccagcgattattgatcaaaatcaaaaggtaatgcacatcctcattcactaagccatgccatgcccaggagactggtttcccggtgacacatccattgctggcaatgagtgtgccagagttattagtgccaagtttttcagaaagtttgaagcaccatggtgtgtcatgctcacttttgtgaaagctgctctgctcagagtctatcaacattgaatatcagttgacagaatggtgccatgcgtggctaacatcctgctttgattccctctgataagctgttctggtggcagtaacatgcaacaaaaatgtgggtgtctctaggcacgggaaacttggttccattgttatattgtcctatgcttcgagccatgggtctacagggtcatccttatgagactcttaaatatacttagatcctggtaagaggcaaagaatcaacagccaaactgctggggctgcaagctgctgaagccagggcatgggattaaagagattgtgcgttcaaacctagggaagcctgtgcccatttgtcctgactgtctgctaacatggtacactgcatctcaagatgtttatctgacacaagtgtattatttctggctttttgaattaatctagaaaatgaaaagatggagttgtattttgacaaaaatgtttgtactttttaatgttatttggaattttaagttctatcagtgacttctgaatccttagaatggcctctttgtagaaccctgtggtatagaggagtatggccactgccccactatttttattttcttatgtaagtttgcatatcagtcatgactagtgcctagaaagcaatgtgatggtcaggatctcatgacattatatttgagtttctttcagatcatttaggatactcttaatctcacttcatcaatcaaatattttttgagtgtatgctgtagctgaaagagtatgtacgtacgtataagactagagagatattaagtctcagtacacttcctgtgccatgttattcagctcactggtttacaaatataggttgtcttgtggttgtaggagcccactgtaacaatattgggcagccttttttttttttttttaattgcaacaatgcaaaagccaagaaagtataagggtcacaagtttaaacaatgaattcttcaacagggaaaacagctagcttgaaaacttgctgaaaaacacaacttgtgtttatggcatttagtaccttcaaataattggctttgcagatattggataccccattaaatctgacagtctcaaatttttcatctcttcaatcactagtcaagaaaaatataaaaacaacaaatacttccatatggagcatttttcagagttttctaacccagtcttatttttctagtcagtaaacatttgtaaaaatactgtttcactaatacttactgttaactgtcttgagagaaaagaaaaatatgagagaactattgtttggggaagttcaagtgatctttcaatatcattactaacttcttccactttttccaaaatttgaatattaacgctaaaggtgtaagacttcagatttcaaattaatctttctatattttttaaatttacagaatattatataacccactgctgaaaaagaaaaaaatgattgttttagaagttaaagtcaatattgattttaaatataagtaatgaaggcatatttccaataactagtgatatggcatcgttgcaatttacagtatcttcaaaaatacagaatttatagaataatttctcctcatttaatatttttcaaaatcaaagttatggtttcctcattttactaaaatcgtattctaattcttcattatagtaaatctatgagcaactccttacttcggttcctctgatttcaaggccatattttaaaaaatcaaaaggcactgtgaactattttgaagaaaacacgacattttaatacagattgaaaggacctcttctgaagctagaaacaatctatagttatacatcttcattaatactgtgttaccttttaaaatagtaattttttacattttcctgtgtaaacctaattgtggtagaaatttttaccaactctatactcaatcaagcaaaatttctgtatattccctgtggaatgtacctatgtgagtttcagaaattctcaaaatacgtgttcaaaaatttctgcttttgcatctttgggacacctcagaaaacttattaacaactgtgaatatgagaaatacagaagaaaataataagccctctatacataaatgcccagcacaattcattgttaaaaaacaaccaaacctcacactactgtatttcattatctgtactgaaagcaaatgctttgtgactattaaatgttgcacatcattcattcactgtatagtaatcattgactaaagccatttgctgtgttttcttcttgtggntgnatatatcaggtaaaatattttccaaagagccatgtgtcatgtaatactgaaccctttgatattgagacattaatttggacccttggtattatctactagaataatgtaatactgnagaaatattgctctaattctttcaaaatggtgcatcccccttaaaangttctatttccataaggatttagcttgcttatcccttcttataccctaagatgaagctgtttttgtgctctttgttcatcattggccctcattccaagcactttacgctgtctgtaatgggatctatttttgcactggaatatctgagaattgcaaaactagacaaaagtttcacaacagatttctaagttaaatcattttcattaaaaggaaaaaagaaaaaaaattttgtatgtcaataactttatatgaagtattaaaatgcatatttctatgttgtaatataatgagtcacaaaataaagctgtgacagttctgttaaaaaaaaaaaaaaaaaaaaaaaaaaaaaaaaaaaaaaaaaaaaaaaaaaaaaaaaaaaaaaaaaaaaaaaaaaaa

SEQ ID NO: 3 - DNA, 10423 bp, Homo sapiens
  misc_feature (9862)..(10423): n=a,t,g or c
agccacggtagaactttctactgtggctctatgctacttcttagcaaccttctccatgtgcttcctggagagtccttggagtcagaacctttttcttgaaacccagacactttacttccaagaaaatgctgtccaagaaaactcatccttcccttcttctcatgaacgttgtgtagagttaatgaccagccacgggcgtccctgctgtgagctctggccgctgccttccagggctcccgagccacacgctgggcgtgctggctgagggaacatggcttgttggcctcagctgaggttgctgctgtggaagaacctcactttcagaagaagacaaacatgtcagctgttactggaagtggcctggcctctatttatcttcctgatcctgatctctgttcggctgagctacccaccctatgaacaacatgaatgccattttccaaataaagccatgccctctgcaggaacacttccttgggttcaggggattatctgtaatgccaacaacccctgtttccgttacccgactcctggggaggctcccggagttgttggaaactttaacaaatccattgtggctcgcctgttctcagatgctcggaggcttcttttatacagccagaaagacaccagcatgaaggacatgcgcaaagttctgagaacattacagcagatcaagaaatccagctcaaacttgaagcttcaagatttcctggtggacaatgaaaccttctctgggttcctatatcacaacctctctctcccaaagtctactgtggacaagatgctgagggctgatgtcattctccacaaggtatttttgcaaggctaccagttacatttgacaagtctgtgcaatggatcaaaatcagaagagatgattcaacttggtgaccaagaagtttctgagctttgtggcctaccaaaggagaaactggctgcagcagagcgagtacttcgttccaacatggacatcctgaagccaatcctgagaacactaaactctacatctcccttcccgagcaaggagctggctgaagccacaaaaacattgctgcatagtcttgggactctggcccaggagctgttcagcatgagaagctggagtgacatgcgacaggaggtgatgtttctgaccaatgtgaacagctccagctcctccacccaaatctaccaggctgtgtctcgtattgtctgcgggcatcccgagggaggggggctgaagatcaagtctctcaactggtatgaggacaacaactacaaagccctctttggaggcaatggcactgaggaagatgctgaaaccttctatgacaactctacaactccttactgcaatgatttgatgaagaatttggagtctagtcctctttcccgcattatctggaaagctctgaagccgctgctcgttgggaagatcctgtatacacctgacactccagccacaaggcaggtcatggctgaggtgaacaagaccttccaggaactggctgtgttccatgatctggaaggcatgtgggaggaactcagccccaagatctggaccttcatggagaacagccaagaaatggaccttgtccggatgctgttggacagcagggacaatgaccacttttgggaacagcagttggatggcttagattggacagcccaagacatcgtggcgtttttggccaagcacccagaggatgtccagtccagtaatggttctgtgtacacctggagagaagctttcaacgagactaaccaggcaatccggaccatatctcgcttcatggagtgtgtcaacctgaacaagctagaacccatagcaacagaagtctggctcatcaacaagtccatggagctgctggatgagaggaagttctgggctggtattgtgttcactggaattactccaggcagcattgagctgccccatcatgtcaagtacaagatccgaatggacattgacaatgtggagaggacaaataaaatcaaggatgggtactgggaccctggtcctcgagctgacccctttgaggacatgcggtacgtctgggggggcttcgcctacttgcaggatgtggtggagcaggcaatcatcagggtgctgacgggcaccgagaagaaaactggtgtctatatgcaacagatgccctatccctgttacgttgatgacatctttctgcgggtgatgagccggtcaatgcccctcttcatgacgctggcctggatttactcagtggctgtgatcatcaagggcatcgtgtatgagaaggaggcacggctgaaagagaccatgcggatcatgggcctggacaacagcatactctggtttagctggttcattagtagcctcattcctcttcttgtgagcgctggcctgctagtggtcatcctgaagttaggaaacctgctgccctacagtgatcccagcgtggtgtttgtcttcctgtccgtgtttgctgtggtgacaatcctgcagtgcttcctgattagcacactcttctccagagccaacctggcagcagcctgtgggggcatcatctacttcacgctgtacctgccctacgtcctgtgtgtggcatggcaggactacgtgggcttcacactcaagatcttcgctagcctgctgtctcctgtggcttttgggtttggctgtgagtactttgccctttttgaggagcagggcattggagtgcagtgggacaacctgtttgagagtcctgtggaggaagatggcttcaatctcaccacttcgatctccatgatgctgtttgacaccttcctctatggggtgatgacctggtacattgaggctgtctttccaggccagtacggaattcccaggccctggtattttccttgcaccaagtcctactggtttggcgaggaaagtgatgagaagagccaccctggttccaaccagaagagaatgtcagaaatctgcatggaggaggaacccacccacttgaagctgggcgtgtccattcagaacctggtaaaagtctaccgagatgggatgaaggtggctgtcgatggcctggcactgaatttttatgagggccagatcacctccttcctgggccacaatggagcggggaagacgaccaccatgtcaatcctgaccgggttgttccccccgacctcgggcaccgcctacatcctgggaaaagacattcgctctgagatgagcaccatccggcagaacctgggggtctgtccccagcataacgtgctgtttgacatgctgactgtcgaagaacacatctggttctatgcccgcttgaaagggctctctgagaagcacgtgaaggcggagatggagcagatggccctggatgttggtttgccatcaagcaagctgaaaagcaaaacaagccagctgtcaggtggaatgcagagaaagctatctgtggccttggcctttgtcgggggatctaaggttgtcattctggatgaacccacagctggtgtggacccttactcccgcaggggaatatgggagctgctgctgaaataccgacaaggccgcaccattattctctctacacaccacatggatgaagcggacgtcctgggggacaggattgccatcatctcccatgggaagctgtgctgtgtgggctcctccctgtttctgaagaaccagctgggaacaggctactacctgaccttggtcaagaaagatgtggaatcctccctcagttcctgcagaaacagtagtagcactgtgtcatacctgaaaaaggaggacagtgtttctcagagcagttctgatgctggcctgggcagcgaccatgagagtgacacgctgaccatcgatgtctctgctatctccaacctcatcaggaagcatgtgtctgaagcccggctggtggaagacatagggcatgagctgacctatgtgctgccatatgaagctgctaaggagggagcctttgtggaactctttcatgagattgatgaccggctctcagacctgggcatttctagttatggcatctcagagacgaccctggaagaaatattcctcaaggtggccgaagagagtggggtggatgctgagacctcagatggtaccttgccagcaagacgaaacaggcgggccttcggggacaagcagagctgtcttcgcccgttcactgaagatgatgctgctgatccaaatgattctgacatagacccagaatccagagagacagacttgctcagtgggatggatggcaaagggtcctaccaggtgaaaggctggaaacttacacagcaacagtttgtggcccttttgtggaagagactgctaattgccagacggagtcggaaaggattttttgctcagattgtcttgccagctgtgtttgtctgcattgcccttgtgttcagcctgatcgtgccaccctttggcaagtaccccagcctggaacttcagccctggatgtacaacgaacagtacacatttgtcagcaatgatgctcctgaggacacgggaaccctggaactcttaaacgccctcaccaaagaccctggcttcgggacccgctgtatggaaggaaacccaatcccagacacgccctgccaggcaggggaggaagagtggaccactgccccagttccccagaccatcatggacctcttccagaatgggaactggacaatgcagaacccttcacctgcatgccagtgtagcagcgacaaaatcaagaagatgctgcctgtgtgtcccccaggggcaggggggctgcctcctccacaaagaaaacaaaacactgcagatatccttcaggacctgacaggaagaaacatttcggattatctggtgaagacgtatgtgcagatcatagccaaaagcttaaagaacaagatctgggtgaatgagtttaggtatggcggcttttccctgggtgtcagtaatactcaagcacttcctccgagtcaagaagttaatgatgccatcaaacaaatgaagaaacacctaaagctggccaaggacagttctgcagatcgatttctcaacagcttgggaagatttatgacaggactggacaccagaaataatgtcaaggtgtggttcaataacaagggctggcatgcaatcagctctttcctgaatgtcatcaacaatgccattctccgggccaacctgcaaaagggagagaaccctagccattatggaattactgctttcaatcatcccctgaatctcaccaagcagcagctctcagaggtggctctgatgaccacatcagtggatgtccttgtgtccatctgtgtcatctttgcaatgtccttcgtcccagccagctttgtcgtattcctgatccaggagcgggtcagcaaagcaaaacacctgcagttcatcagtggagtgaagcctgtcatctactggctctctaattttgtctgggatatgtgcaattacgttgtccctgccacactggtcattatcatcttcatctgcttccagcagaagtcctatgtgtcctccaccaatctgcctgtgctagcccttctacttttgctgtatgggtggtcaatcacacctctcatgtacccagcctcctttgtgttcaagatccccagcacagcctatgtggtgctcaccagcgtgaacctcttcattggcattaatggcagcgtggccacctttgtgctggagctgttcaccgacaataagctgaataatatcaatgatatcctgaagtccgtgttcttgatcttcccacatttttgcctgggacgagggctcatcgacatggtgaaaaaccaggcaatggctgatgccctggaaaggtttggggagaatcgctttgtgtcaccattatcttgggacttggtgggacgaaacctcttcgccatggccgtggaaggggtggtgttcttcctcattactgttctgatccagtacagattcttcatcaggcccagacctgtaaatgcaaagctatctcctctgaatgatgaagatgaagatgtgaggcgggaaagacagagaattcttgatggtggaggccagaatgacatcttagaaatcaaggagttgacgaagatatatagaaggaagcggaagcctgctgttgacaggatttgcgtgggcattcctcctggtgagtgctttgggctcctgggagttaatggggctggaaaatcatcaactttcaagatgttaacaggagataccactgttaccagaggagatgctttccttaacaaaaatagtatcttatcaaacatccatgaagtacatcagaacatgggctactgccctcagtttgatgccatcacagagctgttgactgggagagaacacgtggagttctttgcccttttgagaggagtcccagagaaagaagttggcaaggttggtgagtgggcgattcggaaactgggcctcgtgaagtatggagaaaaatatgctggtaactatagtggaggcaacaaacgcaagctctctacagccatggctttgatcggcgggcctcctgtggtgtttctggatgaacccaccacaggcatggatcccaaagcccggcggttcttgtggaattgtgccctaagtgttgtcaaggaggggagatcagtagtgcttacatctcatagtatggaagaatgtgaagctctttgcactaggatggcaatcatggtcaatggaaggttcaggtgccttggcagtgtccagcatctaaaaaataggtttggagatggttatacaatagttgtacgaatagcagggtccaacccggacctgaagcctgtccaggatttctttggacttgcatttcctggaagtgttctaaaagagaaacaccggaacatgctacaataccagcttccatcttcattatcttctctggccaggatattcagcatcctctcccagagcaaaaagcgactccacatagaagactactctgtttctcagacaacacttgaccaagtatttgtgaactttgccaaggaccaaagtgatgatgaccacttaaaagacctctcattacacaaaaaccagacagtagtggacgttgcagttctcacatcttttctacaggatgagaaagtgaaagaaagctatgtatgaagaatcctgttcatacggggtggctgaaagtaaagaggaactagactttcctttgcaccatgtgaagtgttgtggagaaaagagccagaagttgatgtgggaagaagtaaactggatactgtactgatactattcaatgcaatgcaattcaatgcaatgaaaacaaaattccattacaggggcagtgcctttgtagcctatgtcttgtatggctctcaagtgaaagacttgaatttagttttttacctatacctatgtgaaactctattatggaacccaatggacatatgggtttgaactcacactttttttttttttttgttcctgtgtattctcattggggttgcaacaataattcatcaagtaatcatggccagcgattattgatcaaaatcaaaaggtaatgcacatcctcattcactaagccatgccatgcccaggagactggtttcccggtgacacatccattgctggcaatgagtgtgccagagttattagtgccaagtttttcagaaagtttgaagcaccatggtgtgtcatgctcacttttgtgaaagctgctctgctcagagtctatcaacattgaatatcagttgacagaatggtgccatgcgtggctaacatcctgctttgattccctctgataagctgttctggtggcagtaacatgcaacaaaaatgtgggtgtctctaggcacgggaaacttggttccattgttatattgtcctatgcttcgagccatgggtctacagggtcatccttatgagactcttaaatatacttagatcctggtaagaggcaaagaatcaacagccaaactgctggggctgcaagctgctgaagccagggcatgggattaaagagattgtgcgttcaaacctagggaagcctgtgcccatttgtcctgactgtctgctaacatggtacactgcatctcaagatgtttatctgacacaagtgtattatttctggctttttgaattaatctagaaaatgaaaagatggagttgtattttgacaaaaatgtttgtactttttaatgttatttggaattttaagttctatcagtgacttctgaatccttagaatggcctctttgtagaaccctgtggtatagaggagtatggccactgccccactatttttattttcttatgtaagtttgcatatcagtcatgactagtgcctagaaagcaatgtgatggtcaggatctcatgacattatatttgagtttctttcagatcatttaggatactcttaatctcacttcatcaatcaaatattttttgagtgtatgctgtagctgaaagagtatgtacgtacgtataagactagagagatattaagtctcagtacacttcctgtgccatgttattcagctcactggtttacaaatataggttgtcttgtggttgtaggagcccactgtaacaatattgggcagccttttttttttttttttaattgcaacaatgcaaaagccaagaaagtataagggtcacaagtttaaacaatgaattcttcaacagggaaaacagctagcttgaaaacttgctgaaaaacacaacttgtgtttatggcatttagtaccttcaaataattggctttgcagatattggataccccattaaatctgacagtctcaaatttttcatctcttcaatcactagtcaagaaaaatataaaaacaacaaatacttccatatggagcatttttcagagttttctaacccagtcttatttttctagtcagtaaacatttgtaaaaatactgtttcactaatacttactgttaactgtcttgagagaaaagaaaaatatgagagaactattgtttggggaagttcaagtgatctttcaatatcattactaacttcttccactttttccaaaatttgaatattaacgctaaaggtgtaagacttcagatttcaaattaatctttctatattttttaaatttacagaatattatataacccactgctgaaaaagaaaaaaatgattgttttagaagttaaagtcaatattgattttaaatataagtaatgaaggcatatttccaataactagtgatatggcatcgttgcaatttacagtatcttcaaaaatacagaatttatagaataatttctcctcatttaatatttttcaaaatcaaagttatggtttcctcattttactaaaatcgtattctaattcttcattatagtaaatctatgagcaactccttacttcggttcctctgatttcaaggccatattttaaaaaatcaaaaggcactgtgaactattttgaagaaaacacgacattttaatacagattgaaaggacctcttctgaagctagaaacaatctatagttatacatcttcattaatactgtgttaccttttaaaatagtaattttttacattttcctgtgtaaacctaattgtggtagaaatttttaccaactctatactcaatcaagcaaaatttctgtatattccctgtggaatgtacctatgtgagtttcagaaattctcaaaatacgtgttcaaaaatttctgcttttgcatctttgggacacctcagaaaacttattaacaactgtgaatatgagaaatacagaagaaaataataagccctctatacataaatgcccagcacaattcattgttaaaaaacaaccaaacctcacactactgtatttcattatctgtactgaaagcaaatgctttgtgactattaaatgttgcacatcattcattcactgtatagtaatcattgactaaagccatttgctgtgttttcttcttgtggntgnatatatcaggtaaaatattttccaaagagccatgtgtcatgtaatactgaaccctttgatattgagacattaatttggacccttggtattatctactagaataatgtaatactgnagaaatattgctctaattctttcaaaatggtgcatcccccttaaaangttctatttccataaggatttagcttgcttatcccttcttataccctaagatgaagctgtttttgtgctctttgttcatcattggccctcattccaagcactttacgctgtctgtaatgggatctatttttgcactggaatatctgagaattgcaaaactagacaaaagtttcacaacagatttctaagttaaatcattttcattaaaaggaaaaaagaaaaaaaattttgtatgtcaataactttatatgaagtattaaaatgcatatttctatgttgtaatataatgagtcacaaaataaagctgtgacagttctgttaaaaaaaaaaaaaaaaaaaaaaaaaaaaaaaaaaaaaaaaaaaaaaaaaaaaaaaaaaaaaaaaaaaaaaaaaaaa

SEQ ID NO: 4 - DNA, 16 bp, Homo sapiens
  misc_feature (7)..(10): n=a,t,g or c
aggtcannnnaggtca

SEQ ID NO: 5 - DNA, 16 bp, Homo sapiens
aggttactatcggtca

SEQ ID NO: 6 - DNA, 16 bp, Homo sapiens
ggatcacctgaggtca

SEQ ID NO: 7 - DNA, 16 bp, Homo sapiens
agatcacttgaggtca

SEQ ID NO: 8 - DNA, 16 bp, Homo sapiens
aggttactgaaggcca

SEQ ID NO: 9 - DNA, 16 bp, Homo sapiens
ggatcacctgaggtca

SEQ ID NO: 10 - DNA, 16 bp, Homo sapiens
tgaccgatagtaacct

SEQ ID NO: 11 - DNA, 253 bp, Homo sapiens
tggagagcagcttctgtatgagaaaaatctagctaattatttatttcagtgtctctggaatgcaagctctgtcctgagccacttagaaaacaatttgggatgacaagcatgtgtctcacaatgctgctctggttgccagtgctgtgctgccagttgtcatctttgaacaaactgatgcagtgctggtttaactcttcctctttttggagtaagaaactttggaggcctgtgtccttctagaagtttgctgagc

SEQ ID NO: 12 - DNA, 214 bp, Homo sapiens
cagaaacccacaatggatttgctagaaataatggaacgtcctgtttggacaggatataaccatttctcagctagaggatattgttggaatgaagaaagataaatggggagaagggaactcacattgctttggcacttaaattaagccatgtactgtgttgggaaattatttatattatctcgttgaatccacagtagaacacagttgaacacca

SEQ ID NO: 13 - DNA, 285 bp, Homo sapiens
gtattgatcattgtgttctgagatgggggagcaaattgtgcaggaggccaggtcagtgccaaggtgggtgggaggaattggaggaggaagcttgcctaagtgtgcccagcaaagccacggtagaactttctactgtggctctatgctacttcttagcaaccttctccatgtgcttcctggagagtccttggagtcagaacctttttcttgaaacccagacactttacttccaagaaaatgctgtccaagaaaactcatccttcccttcttctcatgaacgttgtg

SEQ ID NO: 14 - DNA, 4 bp, Homo sapiens
caat

SEQ ID NO: 15 - DNA, 4 bp, Homo sapiens
tata

SEQ ID NO: 16 - DNA, 5 bp, Homo sapiens
tataa

(19) United States
(12) Patent Application Publication
(10) Pub. No.: US 2003/0092157 A1
(43) Pub. Date: May 15, 2003

(54) Compositions, screening systems and methods for modulating HDL cholesterol and triglyceride levels

(76) Inventors: Michael R. Hayden, Vancouver (CA); Roshni R. Singaraja, Vancouver (CA)

Correspondence Address: Alan J. Grant, Esq., c/o Carella, Byrne, Bain, Gilfillan Cecchi,, Stewart & Olstein, 6 Becker Farm Road, Roseland, NJ, 07068, US

(21) Appl. No.: 10/098,939

(22) Filed: Mar. 15, 2002

Related U.S. Application Data
(60) Provisional application No. 60/276,387, filed on Mar. 16, 2001.

(51) Int. Cl. C07K 14/435; C12P 21/02; C12N 5/06; C07H 21/04; C12N 9/16

(52) U.S. Cl. 435/196; 435/69.1; 435/320.1; 435/325; 530/350; 536/23.5

(57) ABSTRACT

The invention features novel ABC1 splice variants and transgenic mice expressing human ABC1 which are useful for studying ABC1 expression, localization, and activity in vivo. Additionally, these ABC1 splice variants and transgenic mice are useful in screening for compounds that modulate cholesterol and triglyceride levels.

This application claims priority of U.S. Provisional Application Serial No. 60/276,387, filed Mar. 16, 2001, the disclosure of which is hereby incorporated by reference in its entirety.

### FIELD OF THE INVENTION

The present invention relates to the field of transgenic animals incorporating selected sequences, such as mutated exons of human genes, and the use of such animals in assays for chemical agents that elevate plasma HDL levels.

### BACKGROUND OF THE INVENTION

Low HDL cholesterol (HDL-C), or hypoalphalipoproteinemia, is a blood lipid abnormality which correlates with a high risk of cardiovascular disease (CVD), in particular coronary artery disease (CAD), but also cerebrovascular disease, coronary restenosis, and peripheral vascular disease. HDL-C levels are influenced by both environmental and genetic factors.

Epidemiological studies have consistently demonstrated that plasma HDL-C concentration is inversely related to the incidence of CAD. HDL-C levels are a strong graded and independent cardiovascular risk factor. Protective effects of an elevated HDL-C persist until 80 years of age. A low HDL-C is associated with an increased CAD risk even with normal (<5.2 mmol/l) total plasma cholesterol levels. Coronary disease risk is increased by 2% in men and 3% in women for every 1 mg/dL (0.026 mmol/l) reduction in HDL-C and in the majority of studies this relationship is statistically significant even after adjustment for other lipid and non-lipid risk factors. Decreased HDL-C levels are the most common lipoprotein abnormality seen in patients with premature CAD. Four percent of patients with premature CAD have an isolated form of decreased HDL-C levels with no other lipoprotein abnormalities while 25% have low HDL-C levels with accompanying hypertriglyceridemia.

Even in the face of other dyslipidemias or secondary factors, HDL-C levels are important predictors of CAD. In a cohort of diabetics, those with isolated low HDL-C had a 65% increased death rate compared to diabetics with normal HDL-C levels (>0.9 mmol/l). Furthermore, it has been shown that even within high risk populations, such as those with familial hypercholesterolemia, HDL-C level is an important predictor of CAD. Low HDL-C levels thus constitute a major, independent, risk for CAD.

These findings have led to increased attention to HDL-C levels as a focus for treatment, following the recommendations of the National Cholesterol Education Program. These guidelines suggest that HDL-C values below 0.9 mmol/l confer a significant risk for men and women. As such, nearly half of patients with CAD would have low HDL-C. It is therefore crucial that we obtain a better understanding of factors which contribute to this phenotype. In view of the fact that pharmacological intervention of low HDL-C levels has so far proven unsatisfactory, it is also important to understand the factors that regulate these levels in the circulation as this understanding may reveal new therapeutic targets.

Absolute levels of HDL-C may not always predict risk of CAD. In the case of CETP deficiency, individuals display an increased risk of developing CAD, despite increased HDL-C levels. What seems to be important in this case is the functional activity of the reverse cholesterol transport pathway, the process by which intracellular cholesterol is trafficked out of the cell to acceptor proteins such as ApoAl or HDL. Other important genetic determinants of HDL-C levels, and its inverse relation with CAD, may reside in the processes leading to HDL formation and intracellular cholesterol trafficking and efflux. To date, this process is poorly understood, however, and clearly not all of the components of this pathway have been identified. Thus, defects preventing proper HDL-mediated cholesterol efflux may be important predictors of CAD. Therefore it is critical to identify and understand novel genes involved in the intracellular cholesterol trafficking and efflux pathways.

HDL particles are central to the process of reverse cholesterol transport and thus to the maintenance of tissue cholesterol homeostasis. This process has multiple steps which include the binding of HDL to cell surface components, the acquisition of cholesterol by passive absorption, the esterification of this cholesterol by LCAT and the subsequent transfer of esterified cholesterol by CETP, to VLDL and chylomicron remnants for liver uptake. Each of these steps is known to impact the plasma concentration of HDL.

Changes in genes for ApoAl-Clll, lipoprotein lipase, CETP (cholesteryl ester transfer protein), hepatic lipase, and LCAT (lecithin cholesterol acyltransferase) all contribute to determination of HDL-C levels in humans. One rare form of genetic HDL deficiency is Tangier disease (TD), diagnosed in approximately 40 patients world-wide, and associated with almost complete absence of HDL-C levels (listed in OMIM as an autosomal recessive trait (OMIM 205400 where OMIM”=“On-Line Mendelian Inheritance in Man” and can be reached at the OMIM Website, a service of the National Center for Biotechnology Information). These patients have very low HDL-C and ApoAl levels, which have been ascribed to impairment of lipid transport and hypercatabolism of nascent HDL and ApoAl, due to a delayed acquisition of lipid and resulting failure of conversion to mature HDL. TD patients accumulate cholesterol esters in several tissues, resulting in characteristic features, such as enlarged yellow tonsils, corneal opacity, hepatosplenomegaly, peripheral neuropathy, and cholesterol ester deposition in the rectal mucosa. Defective removal of cellular cholesterol and phospholipids by ApoAl as well as a marked deficiency in HDL mediated efflux of intracellular cholesterol has been demonstrated in TD fibroblasts. Even though this is a rare disorder, defining its molecular basis could identify pathways relevant for cholesterol regulation in the general population. The decreased availability of free cholesterol for efflux in the surface membranes of cells in Tangier Disease patients appears to be due to a defect in cellular lipid metabolism or trafficking. Approximately 45% of Tangier patients have signs of premature CAD, suggesting a strong link between decreased cholesterol efflux, low HDL-C and CAD. As cholesterol is observed in the rectal mucosa of persons with TD, the molecular mechanism responsible for TD may also regulate cholesterol adsorption from the gastrointestinal (GI) tract.

A more common form of genetic HDL deficiency occurs in patients who have low plasma HDL-C usually below the 5th percentile for age and sex (OMIM 10768), but an absence of clinical manifestations specific to Tangier disease (Marcil et al., Arterioscler. Thromb. Vasc. Biol. 19:159-169, 1999; Marcil et al., Arterioscler. Thromb. Vasc. Biol. 15:1015-1024, 1995). These patients have no obvious environmental factors associated with this lipid phenotype, and do not have severe hypertriglyceridemia nor have known causes of severe HDL deficiency (mutations in ApoAl, LCAT, or LPL deficiency) and are not diabetic. The pattern of inheritance of this condition is most consistent with a Mendelian dominant trait (OMIM 10768).

The development of drugs that regulate cholesterol metabolism has so far progressed slowly. Thus, there is a need for a better understanding of the genetic components of the cholesterol efflux pathway. Newly-discovered components can then serve as targets for drugs.

### BRIEF SUMMARY OF THE INVENTION

The present invention relates to transgenic mice, preferably formed using BACs (bacterial artificial chromosomes) containing a promoter identified as an internal promoter in human intron 1 which contains functional LXREs (specific response elements that bind heterodimers of LXR (liver X receptor) and RXR (retinoid X receptor) and which directly contribute to the regulation of the human ABC1 gene in multiple tissues. The resulting increased human ABC1 protein expression produces a significant increase in cholesterol efflux in different tissues, and marked elevation in HDL-C levels associated with increases in ApoAl and ApoAll.

The present invention also provides three novel ABC1 transcripts that utilize sequences in exon 1. In BAC (bacterial artificial chromosome) transgenic mice there is an increased expression of ABC1 protein, but normal distribution of the ABC1 product in different cells. In accordance with the present invention, intron 1 has an internal promoter and contains functional LXREs in vivo and provides validation for the development of therapeutics influencing ABC1 protein expression.

Thus, the present invention provides ABC1 mRNA splice variants. In one embodiment, the mRNA includes exon 1b, exon 1c, or exon 1d (the sequences of which are listed in bold in FIG. 4A) as well as any ABC1 mRNA that does not contain the previously reported exon 1 (the 303 base pair exon located 24459 base pairs upstream of exon 2) but includes at least one exon selected from the group of exons 2-50. Other mRNA splice variants of the invention do not contain the previously reported exon 1 but include exons 2-50. Still other mRNA splice variants of the invention have equal to or greater than 50, 75, 100, 120, 125, 136, 150, or 178 consecutive nucleotides corresponding to a region of intron 1 of the ABC1 gene, such as nucleotides in exon 1 b, exon 1c, or exon 1d. The invention also provides DNA molecules corresponding to any of the ABC1 mRNA splice variants of the present invention.

These ABC1 mRNA splice variants and the corresponding DNA sequences of the present invention are useful in any of the screen assays involving ABC1 nucleic acid molecules or cells expressing ABC1 nucleic acid molecules (such as those that are described in PCT Publication WO 00/55318 (Sep. 21, 2000), the disclosure of which is incorporated herein by reference in its entirety).

In specific embodiments of the present invention, an ABC1 mRNA splice variant (or fragment thereof or a cell expressing an ABC1 mRNA splice variant (or fragment thereof) is used in screening assays to identify compounds that bind to the mRNA splice variant, modulate the stability of the mRNA splice variant, modulate the rate of translation of the mRNA splice variant, or modulate the rate of RNA processing of the mRNA splice variant. Compounds identified in these assays are useful for the treatment or prevention of a lower than normal HDL cholesterol level, a higher than normal triglyceride level, or a cardiovascular disease in humans or other animals. Compounds that increase the half-life or increase the rate of translation of a mRNA splice variant are useful for increasing HDL cholesterol levels, decreasing triglyceride levels, and preventing and treating cardiovascular disease in humans.

Additionally, the nucleic acids of the present invention are useful in diagnostic methods that involve measuring the amount of an ABC1 mRNA splice variant that is present in a sample from a subject (e.g., a human). The amount of an ABC1 mRNA splice variant may be correlated with a subject's risk for developing a lower than normal HDL cholesterol level, a higher than normal triglyceride level, or a cardiovascular disease. Alternatively, the amount of an ABC1 mRNA splice variant may be used to diagnosis a subject with a lower than normal HDL cholesterol level, a higher than normal triglyceride level, or a cardiovascular disease.

In another aspect, the invention features an expression vector, a cell, or a non-human mammal, preferably a mouse, that includes a nucleic acid molecule of the present invention.

In a related aspect, the invention features a cell from a non-human mammal having a transgene that includes a nucleic acid molecule of the present invention.

In yet another aspect, the invention features a transgenic non-human mammal expressing an ABC1 mRNA or protein from another genus or species (e.g., human ABC1 mRNA or protein). For example, the invention includes a transgenic mouse expressing a human ABC1 mRNA or protein. The ABC1 mRNA expressed by the transgenic animal may be the previously described ABC1 mRNA that includes exon 1 or may be any of the ABC1 mRNA splice variants of the present invention. The transgenic animal may express endogenous ABC1 mRNA or protein in addition to the heterologous ABC1 mRNA or protein, or the transgenic animal may only express heterologous ABC1 mRNA or protein.

These transgenic animals are useful for the in vivo study of ABC1 mRNA and protein expression, localization, and activity. These transgenic animals are also useful as animal models for the testing of candidate compounds that modulate cholesterol or triglyceride levels in vivo. For example, these transgenic animals may be used to identify candidate compounds that increase HDL cholesterol levels or that decrease triglyceride levels and thus are useful as therapeutics for the treatment or prevention of a lower than normal HDL cholesterol level, a higher than normal triglyceride level, or a cardiovascular disease in humans or other animals. Alternatively, these transgenic animals may be used to test candidate compounds that are useful for the treatment or prevention of any disease or condition to determine whether they may cause undesirable side-effects, such as the lowering of HDL cholesterol levels or the raising of triglyceride levels in humans. Compounds that cause these adverse side-effects may be eliminated from drug development or chemically modified to generate related compounds with less ability to lower HDL cholesterol levels or raise triglyceride levels.

A preferred screening method involves administering a candidate compound to a transgenic animal expressing a heterologous ABC1 mRNA or protein and measuring the level of heterologous ABC1 mRNA, heterologous ABC1 protein, cholesterol efflux, or plasma lipid. The candidate compound is determined to modulate ABC1 activity or modulate cholesterol or triglyceride levels if the candidate compound effects a change in the level of heterologous ABC1 mRNA, heterologous ABC1 protein, cholesterol efflux, or plasma lipid.

The transgenic animals of the present invention may also be used in any of the assays described in PCT Publication WO 00/55318, published Sep. 21, 2000.

### DEFINITIONS

By “substantially identical” is meant a polypeptide or nucleic acid exhibiting at least 50%, preferably 85%, more preferably 90%, and most preferably 95% identity to a reference amino acid or nucleic acid sequence. For polypeptides, the length of comparison sequences will generally be at least 16 amino acids, preferably at least 20 amino acids, more preferably at least 25 amino acids, and most preferably 35 amino acids. For nucleic acids, the length of comparison sequences will generally be at least 50 nucleotides, preferably at least 60 nucleotides, more preferably at least 75 nucleotides, and most preferably 110 nucleotides. One sequence may include additions or deletions (i.e., gaps) of 20% or less when compared to the second sequence. Optimal alignment of sequences may be conducted, for example, by the methods of Gish and States (Nature Genet. 3:266-272, 1993), Altshul et al. (J. Mol. Biol. 215:403-410, 1990), Madden et al. (Meth. Enzymol. 266:131-141, 1996), Althsul et al (Nucleic Acids Res. 25:3389-3402, 1997), or Zhang et al (Genome Res. 7:649-656,1997).

As used herein, the term “percent identity” or “percent identical,” when referring to a sequence, means that a sequence is compared to a claimed or described sequence after alignment of the sequence to be compared (the “Compared Sequence”) with the described or claimed sequence (the “Reference Sequence”). The Percent Identity is then determined according to the following formula:

Percent Identity=100[1−(C/R)]

wherein C is the number of differences between the Reference Sequence and the Compared Sequence over the length of alignment between the Reference Sequence and the Compared Sequence wherein (i) each base or amino acid in the Reference Sequence that does not have a corresponding aligned base or amino acid in the Compared Sequence and (ii) each gap in the Reference Sequence and (iii) each aligned base or amino acid in the Reference Sequence that is different from an aligned base or amino acid in the Compared Sequence, constitutes a difference; and R is the number of bases or amino acids in the Reference Sequence over the length of the alignment with the Compared Sequence with any gap created in the Reference Sequence also being counted as a base or amino acid.

If an alignment exists between the Compared Sequence and the Reference Sequence for which the percent identity as calculated above is about equal to or greater than a specified minimum Percent Identity then the Compared Sequence has the specified minimum percent identity to the Reference Sequence even though alignments may exist in which the hereinabove calculated Percent Identity is less than the specified Percent Identity.

Sequence identity is typically measured using sequence analysis software with the default parameters specified therein (e.g., Sequence Analysis Software Package of the Genetics Computer Group, University of Wisconsin Biotechnology Center, 1710 University Avenue, Madison, Wis. 53705). This software program matches similar sequences by assigning degrees of homology to various substitutions, deletions, and other modifications.

By “substantially pure nucleic acid” is meant nucleic acid that is free of the genes which, in the naturally-occurring genome of the organism from which the nucleic acid of the invention is derived, flank the nucleic acid. The term therefore includes, for example, a recombinant nucleic acid that is incorporated into a vector; into an autonomously replicating plasmid or virus; into the genomic nucleic acid of a prokaryote or a eukaryote cell; or that exists as a separate molecule (e.g., a cDNA or a genomic or cDNA fragment produced by PCR or restriction endonuclease digestion) independent of other sequences. It also includes a recombinant nucleic acid that is part of a hybrid gene encoding additional polypeptide sequence.

By “ABC1 polypeptide” is meant a polypeptide having substantial identity to the previously reported amino acid sequence of human ABC1 (PCT Publication WO 00/55318, published Sep. 21, 2000).

By “ABC biological activity” or “ABC1 biological activity” is meant hydrolysis or binding of ATP, transport of a compound (e.g., cholesterol, interleukin-1) or ion across a membrane, or regulation of cholesterol or phospholipid levels (e.g., either by increasing or decreasing HDL cholesterol or LDL-cholesterol levels).

By “modulates” is meant increase or decrease. Preferably, a compound that modulates LXR-mediated transcription, RXR-mediated transcription, ABC1 gene expression, HDL-C levels, or triglyceride levels does so by at least 5%, more preferably by at least 10%, and most preferably by at least 25% or even at least 50%.

As used herein, and in keeping with the latest nomenclature, ABC1 is now termed ABCA1 and so these terms will be considered the same unless expressly stated to be otherwise. The term “ABC1” refers to ATP-binding cassette transporter 1.

### BRIEF DESCRIPTION OF THE DRAWINGS

FIGS. 1A and 1B are schematic illustrations of the localization of LXRE elements in the ABC1 5′ region. FIG. 1A shows the putative LXRE elements discovered in intron 1 are shown. ABC1 genomic organization at the 5′ end is shown above, and the ABC1 BAC269 is shown below. As illustrated in FIG. 1B, the BAC contains 13.5 kb of intron 1 sequence followed by the rest of the gene, with the ATG occurring in exon 2. Novel putative LXR elements were identified at positions −11050 bp (SEQ ID NO: 6), −7670 bp (SEQ ID NO: 8), −7188 bp (SEQ ID NO: 7) and −4696 bp (SEQ ID NO: 9) in the ABC1 genomic DNA and are also contained within the BAC (A and B). The sequence at +4 is SEQ ID NO: 10.

FIGS. 2A and 2B are a bar graph and picture of a gel demonstrating that intron 1 of ABC1 has promoter activity. For FIG. 2A, HepG2 (liver), HuH7 (hepatoma), CaCo₂ (intestinal) and RK13 (kidney) cell lines were cotransfected with empty pGI3 vector or pGI3 containing an 8 kb fragment upstream of exon 2 of ABC1 intron 1 (pGI3-8 kb). Cells were then incubated for 48 hour. Luciferase activity was determined and plotted as fold activation relative to empty pGI3-transfected cells. The results of gel mobility shift assays in which LXRα and RXR were incubated as indicated with the radiolabeled probe corresponding to CYP7-LXRE are shown in FIG. 2B. Binding of the LXRα-RXR heterodimer was tested by competition, by adding 5-, 25-, or 50-fold molar excess of each unlabeled oligonucleotide corresponding to the putative LXREs shown in FIG. 1 and Table 1 (the latter shows the position and sequence of LXR elements in the human ABC1 gene).

FIG. 3 is a bar graph illustrating that the LXREs are functional for LXRα transactivation. Cos-1 cells were cotransfected with multiple copies of the putative LXREs in the reporter plasmid TKpGI3 and expression plasmids for LXRα and RXR. Cells were then treated for 48 h with 1 μM 22(R)-hydroxycholesterol. Luciferase activity was determined and plotted as fold activation relative to vehicle-treated cells.

FIG. 4A is a schematic diagram indicating the location of the splice variants discovered in the BAC transgenic mice and also confirmed in wild type mice and humans. Exon 1b occurs 1742bp upstream of exon 2, and is 120 bp in length. Exon 1b contains a TA rich region upstream, approximately 2.5 kb upstream. Exon 1c is 136 bp in length, and exon 1d 178 bp in length. A CAAT box is located upstream of exon 1b, and a CAAT (SEQ ID NO: 14) box and a TATA (SEQ ID NO: 15) box are immediately upstream of exon 1c. FIG. 4A also lists a portion of the intronic sequence upstream of exons 1b, exon 1c, and exon 1d. The intronic sequence is listed in regular, unbolded font, with the exception that the CAAT (SEQ ID NO: 14) and TATAA (SEQ ID NO: 16) sequences are bolded. The remainder of the bolded sequence represents the sequences of exons 1b, exon 1c, and exon 1d, respectively. FIG. 4B is a picture of a gel illustrating the identification of the alternative transcript involving exon 1 B in wild type and BAC mouse liver tissue. Duplex RT-PCR was performed on mouse RNA with primers generating an alternative transcript fragment of ˜360 bp and a fragment corresponding to the previously characterized transcript of ˜250 bp. Both transcripts were found in wild type and transgenic mice, with the alternative transcript being highly upregulated in the BAC transgenic mice. Exons E1c and E1d are also shown. The sequence shown as including E1b is SEQ ID NO: 11, the one including E1c is SEQ ID NO: 12, and the one including E1d is SEQ ID NO: 13.

FIGS. 5A-5C are pictures of Western blot analysis showing the expression of ABC1 in BAC transgenic mouse tissue. FIG. 5A is a picture of the Western blot analysis of various mouse tissues from BAC transgenic mice and control littermates on a chow diet. Tissue from two different founder lines were analyzed and showed similar results. ABC1 was detected in liver, brain, small intestine, testis, lung and stomach, with highest level of up-regulation in the BAC mouse liver when compared to control. FIG. 5B shows the up-regulation of ABC1 protein levels in liver in response to feeding of an atherogenic diet for 7 days ad libitum. There was a graded increase in ABC1 protein levels, with liver from wild type chow fed animals showing the lowest levels, and transgenic animals fed the atherogenic diet showing the highest levels. All western blots were probed with an anti GAPDH antibody (Sigma) to ensure equal protein loading levels, and the corresponding GAPDH lanes are shown below the western blots. FIG. 5C is a picture of a Western blot performed on cultured macrophage and fibroblast cells that were used for efflux assays. ABC1 protein was detected in both peritoneal macrophage and fibroblast cells, with the transgenic animals showing higher levels of protein that the control littermates. The protein levels in these tissues were also upregulated in response to feeding of an atherogenic diet.

FIGS. 6A-M are pictures of the immunocytochemical analysis illustrating the distribution of ABC1 protein in wildtype and ABC1 BAC transgenic mice. Localization of ABC1 protein was determined by immunocytochemical analysis using an ABC1 specific polyclonal antibody (ABCpep4) in liver and brain tissues. Endogenous levels of ABC1 are identified in sections from wildtype liver (low power, FIG. 6A and high power FIG. 6C), elevated ABC1 levels are seen in liver tissues from BAC transgenics (low power, FIG. 6B and high power, FIG. 6D). The tissue distribution of ABC1 is similar in sections from cerebral cortex from both wild-type and BAC transgenic brains (FIGS. 6F and G). ABC1 was predominantly neuronal as shown by co-localization with the neuronal marker NeuN in wildtype (FIGS. 6H, I, and J) and BAC transgenic brains (FIGs. 6K, L, and M).

FIGS. 7A and 7B are graphs of the analysis of plasma lipid levels in transgenic mice. As illustrated in FIG. 7A, BAC transgenic mice show a 65% increase in HDL-C levels compared to control littermates. Furthermore, both HDL-C levels in both BAC transgenic and wild type mice were upregulated by >100% in response to feeding of the atherogenic diet, with the BAC transgenic mice having close to 2× the HDL-C seen in their nontransgenic littermates. While quantitative changes are apparent, there are no obvious qualitative changes in HDL-C in transgenic versus nontransgenic mice, either on chow (FIG. 7B left panel) or atherogenic diets (FIG. 7B right panel).

FIGS. 8A and 8B are bar graphs showing the analysis of efflux levels in primary macrophage and fibroblast cells. For FIG. 8A, mice (n=4, and see Table 3, which shows an analysis of [3H]-cholesterol efflux in ApoAl in ABC1 BAC transgenic mice) were used to establish primary macrophage cultures as described. Efflux was measured 24 hours after the addition of ApoAl and after stimulation by 9(cis) retinoic acid and 22(R)-hydroxy cholesterol. We observed a significant increase (46%) in efflux levels of BAC transgenic macrophages when compared to macrophages from wild type littermate (p<0.01). In both sets of animals similarly efflux increased upon stimulation of the cultures with 9(cis) retinoic acid and 22(R)-hydroxy cholesterol compared to untreated values. BAC transgenic mice on the atherogenic diet showed an increase in efflux of 20.4% when compared to BAC mice on the control chow diet (p<0.01). This efflux rate was further increased when the cultures were stimulated with 9(cis) retinoic acid and 22(R)-hydroxy cholesterol (p<0.01). For FIG. 8B, efflux was also performed on fibroblast cultures established from BAC transgenic and control mice (n=4, and see Table 3, which shows an analysis of [3H]-cholesterol efflux in ApoAl in ABC1 BAC transgenic mice). Increase in efflux to ApoAl of 25.2% in BAC transgenic mice when compared to control liftermates on chow diet is seen (p<0.01). This level is further increased by 55.8% in response to stimulation by 9(cis) retinoic acid and 22(R)-hydroxy cholesterol. BAC transgenic mice fed the atherogenic diet showed a significant (102%) increase in efflux levels when compared to chow diet transgenic animals. These levels were further increased by 11.2% when the cultures were stimulated with 9(cis) retinoic acid and 22(R)-hydroxy cholesterol.

### DETAILED DESCRIPTION OF THE INVENTION

It is now known that the human ABC1 (also known as ABCA1) genomic region contains consensus binding sites for transcription factors such as LXRs, RXRs, PPARs, SREBPs, and RORs (PCT Publication WO 00/55318, published Sep. 21, 2000; U.S. Provisional Application No. 60/213,958 filed Jun. 23, 2000, U.S. Utility Application No. 09/526,193, filed Mar. 15, 2000, U.S. Provisional Application No. 60/124,702, filed Mar. 15, 1999; U.S. Provisional Application No. 60/138,048, filed Jun. 8, 1999; U.S. Provisional Application No. 60/139,600, filed Jun. 17, 1999; U.S. Provisional Application No. 60/151,977, filed Sep. 1, 1999; see also: Human ABCA1 BAC transgenic mice show increased high density lipoprotein cholesterol and ApoAl-dependent efflux stimulated by an internal promoter containing liver X receptor response elements in intron 1, Singaraja RR, et al., J. Biol. Chem., 276 (36):33969-79 (2001), the disclosures of each of which patents, publications and patent applications are incorporated herein by reference in their entirety).

In accordance with the foregoing, by using BAC transgenic mice, increased human ABCA1 protein expression resulted in a significant increase in cholesterol efflux in different tissues and marked elevation in high density lipoprotein (HDL)-cholesterol levels associated with increases in apoAl and apoAll. Three novel ABCA1 transcripts containing three different transcription initiation sites that utilize sequences in intron 1 have been identified. In BAC transgenic mice there is an increased expression of ABCA1 protein, but the distribution of the ABCA1 product in different cells remains similar to wild type mice. An internal promoter in human intron 1 containing liver X response elements (LXR) is functional in vivo and directly contributes to regulation of the human ABCA1 gene in multiple tissues and to raised HDL cholesterol, apoAl, and apoAll levels. A highly significant relationship between raised protein levels, increased efflux, and level of HDL elevation is evident. These data provide proof of the principle that increased human ABCA1 efflux activity is associated with an increase in HDL levels in vivo.

In accordance with the foregoing, the present invention relates to an isolated polynucleotide comprising:

(a) a polynucleotide that encodes a polypeptide having ATP-Binding Cassette transporter 1 (ABCA1) activity wherein said isolated polynucleotide further comprises a sequence selected from the group consisting of exons E1b, E1c and E1d and does not comprise Exon 1 of ABCA1, and

(b) the full complement of (a).

In one preferred embodiment, the polynucleotide of (a) that encodes said polypeptide is at least 95% identical to the sequence of SEQ ID NO: 1, most preferably wherein the polynucleotide of (a) that encodes said polypeptide is identical to the sequence of SEQ ID NO: 1.

In another preferred embodiment, the polynucleotide of (a) that encodes said polypeptide is at least 95% identical to the sequence of SEQ ID NO: 2, most preferably wherein the polynucleotide of (a) that encodes said polypeptide is identical to the sequence of SEQ ID NO: 2.

In an additional preferred embodiment, the polynucleotide of (a) that encodes said polypeptide is at least 95% identical to the sequence of SEQ ID NO: 3, most preferably wherein the polynucleotide of (a) that encodes said polypeptide is identical to the sequence of SEQ ID NO: 3.

In a further preferred embodiment, the present invention includes an isolated polynucleotide comprising a nucleotide sequence selected from the group consisting of SEQ ID NO: 1, 2 and 3 and wherein said isolated polynucleotide does not comprise Exon 1 of ABCA1. As disclosed herein, SEQ ID NO: 1, 2 and 3 each comprise exons 2 to 50 but not exon 1. Each of the sequences differs in having a different splice variant in place of exon 1 (E1B for SEQ ID NO: 1, E1c for SEQ ID NO: 2 and E1d for SEQ ID NO: 3). These sequences represent isolated cDNAs but can also be messenger RNAs.

Also disclosed herein are sequences of additional regions of the ABC1 regulatory region which contain consensus binding sites for transcription factors. Further disclosed herein are heterozygotes for ABC1 mutations having age-modulated decreases in HDL, increases in triglyceride levels, and significantly increased risk for CAD. Furthermore, this phenotype is highly correlated with efflux, clearly demonstrating that impairment of reverse cholesterol transport is associated with decreased plasma HDL cholesterol, increased triglyceride levels, and increased atherogenesis.

A significant step in the elucidation of mechanisms of reverse cholesterol transport resulted from the identifications of mutations in ABC1 underlying Tangier disease, as well as familial hypoalphalipoproteinemia associated with reduced efflux (1-5). These and further investigation and characterization of the biochemical phenotype of heterozygotes for ABC1 deficiency (6), have demonstrated that lipidation of the nascent ApoAl rich HDL particle is a rate limiting step in the maintenance and regulation of HDL cholesterol (HDL-C) levels in humans. The ABC1 gene is also rate limiting for cholesterol efflux and HDL-C levels in different species, including mouse (7,8) and chicken (9), demonstrating conservation of this pathway in cholesterol metabolism over at least 400 million years.

Studies of heterozygotes for ABC1 deficiency have also demonstrated a very strong relationship between levels of cellular cholesterol efflux and HDL-C levels in plasma, with approximately 82% of the variation in HDL-C levels in these patients being accounted for by the decrease in cellular cholesterol efflux. This clearly has demonstrated in these patients that ABC1 is the major, but not the only, important contributor to cellular cholesterol efflux in humans (6).

With respect to regulation of ABC1 expression, the direct mechanism of sterol mediated upregulation of gene expression of ABC1 has been shown to be due to transactivation of the ABC1 promoter by LXR and RXR (10-12), two members of the nuclear receptor superfamily. This sterol mediated activation has been shown to be dependent on the binding of RXR/LXR heterodimers to a DR4 element in the promoter of the ABC1 gene. Transcriptional sequences representing LXR response elements, (or LXREs), are composed of direct repeats of the motif AGGTCA separated by 4 nucleotides (SEQ ID NO: 4), and this element has been shown to be activated by both ligands of RXR (retinoids) and LXR (oxysterols) separately and together. These reports (10-12) have clearly shown that the LXR response element in the promoter influences ABC1 regulation in vitro (see also: Janowski et al, Proc. Natl. Acad. Sci. USA 96:266-271 (1999)). However, further characterization of additional LXREs in the ABC1 gene has not been presented and there has been no in vivo data on the sterol responsiveness of the human ABC1 gene.

Levels of mRNA may be poor predictors of protein expression (12). Messenger RNA levels can vary almost 20× and still yield the same level of a gene product. Alternatively, the same level of expression of an mRNA can result in vastly different levels of a protein (13,14). Therefore, even though in vitro studies have shown an increase of ABC1 mRNA, it is most important to assess the change in ABC1 protein secondary to increased ABC1 mRNA expression. Furthermore, while decreases in cellular cholesterol efflux secondary to either antisense in vitro inhibition of the gene(1) or in vivo mutations (1-5) have been shown to be associated with decreased efflux and decreased HDL-C levels, it is currently unknown whether over-expression of ABC1 in vivo is associated with increased HDL-C levels and an increase in tissue specific cholesterol efflux.

The use of transgenic technologies employing BACs offers important advantages for generating mice expressing human ABC1. The inclusion of endogeneous regulatory elements within the transgene essentially humanizes the mouse in this respect that it allows for assessment of normal temporal, tissue and cell specific expression of human ABC1. Furthermore, inclusion of selected endogenous promoter sequences allows for dissection of the contribution of different sequences to the normal regulation of the ABC1 gene. Such information is not possible using cDNA transgenic approaches which often results in poorly expressed genes that are not physiologically regulated.

As disclosed herein the ABC1 gene has LXREs in intron 1 with demonstrated in vitro and in vivo activity. Increasing human ABC1 protein expression through activation of this functional internal promoter in human intron 1 by oxysterols in vivo, directly contributes to the regulation of the human ABC1 gene in multiple tissues. These experiments have led to the identification of three novel ABC1 transcripts that utilize sequences in intron 1. In addition, increased human ABC1 expression results in a significant increase in cholesterol efflux and HDL-C levels. These studies provide important proof of principle for therapeutic strategies directed to activation of ABC1 expression and activity.

The LXREs in intron 1, which are more than 20 kb away from the promoter, clearly identify the importance of intragenic LXR sequences for the regulation of ABC1. This, to our knowledge, is the first report of intragenic LXR sequences contributing to gene expression. These LXREs are functional in vivo, and alone can significantly raise HDL-C levels and increase ABC1 protein expression, particularly notably in liver, brain, the small intestine, macrophages and fibroblasts. Our experiments also demonstrate cross species functional complementarity with murine LXR-α, β and RXR-α, sufficient to transactivate the human ABC1 gene.

Increasing human ABC1 protein expression results in a significant increase in HDL-C levels. No obvious change in the distribution of HDL cholesterol is seen, suggesting that this increase in ABC1 protein results in an increase in the number of HDL particles, without obvious changes in the nature and lipoprotein composition of these particles. We have previously shown based on families with low HDL, a strong correlation between the reduction in cholesterol efflux and the decreased HDL plasma in these patients (6). Thus, increasing efflux is associated with a proportionate and predictable increase in HDL cholesterol and raised ApoAl and ApoAll levels. The relationship between the increase in efflux and increase in HDL appears to be linear, with a correlation coefficient of 0.87 (p=0.007) showing that in these studies that raised efflux levels are associated directly with a proportionate increase in HDL in vivo. Furthermore, the rate of efflux was almost completely correlated with the level of ABC1 protein (r²=0.98, p=0.001), further showing that any approach which results in an increase in net functional ABC1 protein levels in the cell, would be expected to have a proportionate increase in cholesterol efflux. The establishment of humanized transgenic mice containing sequence from all of the introns, including intron 1 without promoter sequence, has allowed dissection and determination of the contribution of these LXRE elements to the normal regulation and responsiveness to oxysterol stimulation.

At the present time, approximately 35 mutations have been described in the ABC1 gene (1-6). However, we have found that in some patients in whom mutations have been mapped to this particular gene, no DNA sequence variation in the coding region or splice donor/acceptor sites, which could account for the phenotype seen, has been detected. The approach to assessing the mutations had been to look at each splice site and exon, as well as the regular promoter in an effort to identify potential DNA variants that could account for the disruption of protein function(1,6). The failure to detect mutations in some of these patients, together with the finding of the importance of these alternate transcripts in the regulation of the ABC1 gene, may explain how expression could be compromised in some patients with defects in efflux, which map to this gene but in whom no mutations have yet been described. Mutations disrupting the ratios of alternatively spliced protein isoforms have been implicated as the cause of abnormal urogenital development in Denys-Drash syndrome(26). Further analysis and comparison of the sequence of these different ABC1 transcripts may help to identify missing mutations and further confirm the functional significance of these sequences.

The human ABC1 gene comprises 50 exons spanning 149 kb of genomic DNA. Translation begins in exon 2 and transcription had previously been shown to be initiated at a 303 base pair exon located 24459 base pairs upstream of exon 2 (18). Here we have shown three other transcription initiation sites and demonstrated alternate splicing of this transcript utilizing sequence from intron 1. It is apparent that alternate splicing involving sequence in intron 1 can be used to enhance the information contained with the ABC1 gene. Alternate splicing of nuclear pre-mRNA is a general mechanism for controlling gene expression leading to various RNA isoforms from a single primary transcript(27-29). What is unusual here is that the splicing event involves intronic sequence, which in contrast to alternate splicing of exonic sequence, has only been described infrequently(30-32). The specific capacities of these sequences in intron 1 for protein interactions and the importance of the contribution of these specific sequences in modulating cellular responses to physiological signals, such as oxysterol stimulation, when compared to the promoter LXRE's, remains to be determined. However, it is clear that alternate splicing decisions in regard to intron 1 sequence are influenced by specific factors which may vary in different cell types, suggesting this event is of primary importance.

These newly discovered alternate transcripts are not observed equally in all tissues and, therefore, may provide further insights into the complex tissue-specific regulation of this gene, with certain transcripts likely to play a more major role in certain tissues. The presence of this alternate transcript is also seen in mouse tissues, but there appears to species-specific regulation of ABC1 with this transcript not being seen in all tissues (e.g., liver) with the same level as it is seen in humans.

In accordance with the foregoing, the present invention relates to a vector comprising a polynucleotide that encodes an active ABCA1 polypeptide and wherein said polynucleotide comprises a member selected from the group consisting of E1b, E1c and E1d but does not comprise Exon 1 of ABCA1. In a preferred embodiment thereof, the vector is a bacterial artificial chromosome (BAC).

The present invention further relates to a recombinant cell containing such a vector. In a preferred embodiment, said cell is a mammalian cell, most preferably a human cell.

The present invention further relates to a non-human transgenic animal wherein the genome of one or more cells of said animal comprises a polynucleotide that encodes an active ABCA1 polypeptide and wherein said polynucleotide comprises a member selected from the group consisting of E1b, E1c and E1d but does not comprise Exon 1 of ABCA1. In a preferred embodiment, said animal has been produced using a BAC and is therefore a BAC transgenic animal, most preferably a mouse.

Other preferred transgenic animals include chickens (e.g., WHAM chickens), cows, sheep, big-horn sheep, goats, buffaloes, antelopes, oxen, horses, donkeys, mule, deer, elk, caribou, water buffalo, camels, llama, alpaca, rabbits, pigs, rats, guinea pigs, hamsters, and primates such as monkeys. These transgenic animals may be generated using any standard method such as the method described herein.

Species-specific regulation of other genes involved in HDL metabolism has been reported. For example, fibrates decrease the transcription of the ApoAl gene in rats whilst in humans, this clearly results in activation of ApoAl gene expression. The availability of humanized ABC1 transgenic mice further allows the investigation of the role of other transcription factors influencing the responsiveness of this promoter to oxysterol stimulation. The breeding of these mice to others where various transcription factors are no longer present, will help to determine their role influencing the contribution of other factors to the responsiveness of this promoter to oxysterol stimulation.

Furthermore, the availability of these mice facilitates determination of how effectively these animals can resist experimental atherosclerosis. Since the first description of the cellular defect in Tangier disease where decreased HDL levels appeared to be associated with a decrease in cholesterol efflux (33,34), the question as to whether increasing efflux would result in an increase in HDL-C levels has been present. This matter assumed greater importance with the discovery of the ABC1 gene as the gene for Tangier Disease (1-6). The discovery and demonstration that increasing cholesterol efflux can indeed be associated with an increase in HDL-C levels, provides additional support for the development of therapeutics influencing ABC1 protein expression.

In accordance with the foregoing, the present invention further relates to a method for identifying a compound that modulates ABCA1 biological activity comprising:

(a) contacting a compound with a polynucleotide that encodes a polypeptide having ABCA1 activity wherein said polynucleotide further comprises a sequence selected from the group consisting of exons E1b, E1c and E1d and does not comprise Exon 1 of ABCA1 and under conditions promoting said contacting and promoting expression of said polynucleotide, and

(b) determining a difference in expression of said polynucleotide in the presence of said compound compared to when said compound is not present, thereby identifying a compound that modulates ABCA1 biological activity.

In a preferred embodiment of this method, the difference in expression is an increase in expression. In different embodiments thereof, the expression is determined by determining the production of RNA encoded by said polynucleotide or is determined by determining the production of polypeptide encoded by said polynucleotide or by any other of the methods disclosed herein or known in the art..

In a preferred embodiment, the polynucleotides used in the methods of the invention are DNA or RNA, including cDNA. In one preferred embodiment, the polynucleotide is present in a recombinant cell as disclosed herein, especially one created using a BAC.

In another preferred embodiment, the polynucleotide is present in a non-human transgenic animal as disclosed herein.

The present invention further relates to a method for identifying a compound that modulates plasma lipid levels in an animal comprising administering to an animal an effective amount of a compound that modulates the expression of ABCA1 biological activity when such modulation is determined by an assay or screening method of the invention and determining a change in plasma lipid levels as compared to when said compound has not been administered to said animal.

In preferred embodiments, the lipid is a triglyceride, a phospholipid, or cholesterol, especially where the cholesterol is part of HDL-cholesterol.

In another preferred embodiment, the is a mammal, most preferably a human being.

In another preferred embodiment, said animal is a non-human transgenic animal as disclosed herein, most preferably a mouse.

The present invention also relates to a method of lowering plasma lipid level in a mammal exhibiting elevated plasma lipid level comprising administering to said animal an effective amount of a compound first identified as lowering plasma lipid levels using a screening or assay method of the invention. In a preferred embodiment, the mammal is a human being. In other preferred embodiments thereof, the lipid is a triglyceride, a phospholipid, or cholesterol, most preferably HDL-cholesterol.

The agents and compounds identified as modulators of plasma lipid levels according to the present invention are typically used in the form of a composition in which the agent or compound is present in a pharmaceutically acceptable carrier. Methods well known in the art for making formulations are found in, for example, Remington: The Science and Practice of Pharmacy, (19th ed.) ed. A. R. Gennaro A R., 1995, Mack Publishing Company, Easton, Pa. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for agonists of the invention include ethylenevinyl acetate copolymer particles, osmotic pumps, implantable infusion systems, and liposomes. Formulations for inhalation may contain excipients, or example, lactose, or may be aqueous solutions containing, for example, polyoxyethylene-9-lauryl ether, glycocholate and deoxycholate, or may be oily solutions for administration in the form of nasal drops, or as a gel.

The present invention also relates to a process that comprises a method for producing a product comprising identifying an agent according to one of the disclosed processes for identifying such an agent (i.e., the therapeutic agents identified according to the assay procedures disclosed herein) wherein said product is the data collected with respect to said agent as a result of said identification process, or assay, and wherein said data is sufficient to convey the chemical character and/or structure and/or properties of said agent. For example, the present invention specifically contemplates a situation whereby a user of an assay of the invention may use the assay to screen for compounds having the desired enzyme modulating activity and, having identified the compound, then conveys that information (i.e., information as to structure, dosage, etc) to another user who then utilizes the information to reproduce the agent and administer it for therapeutic or research purposes according to the invention. For example, the user of the assay (user 1) may screen a number of test compounds without knowing the structure or identity of the compounds (such as where a number of code numbers are used the first user is simply given samples labeled with said code numbers) and, after performing the screening process, using one or more assay processes of the present invention, then imparts to a second user (user 2), verbally or in writing or some equivalent fashion, sufficient information to identify the compounds having a particular modulating activity (for example, the code number with the corresponding results). This transmission of information from user 1 to user 2 is specifically contemplated by the present invention.

The invention is described in more detail in the following non-limiting examples. It is to be understood that these methods and examples in no way limit the invention to the embodiments described herein and that other embodiments and uses will no doubt suggest themselves to those skilled in the art.

### EXAMPLE 1

### Experimental Procedures

### Transient Transfection Assay

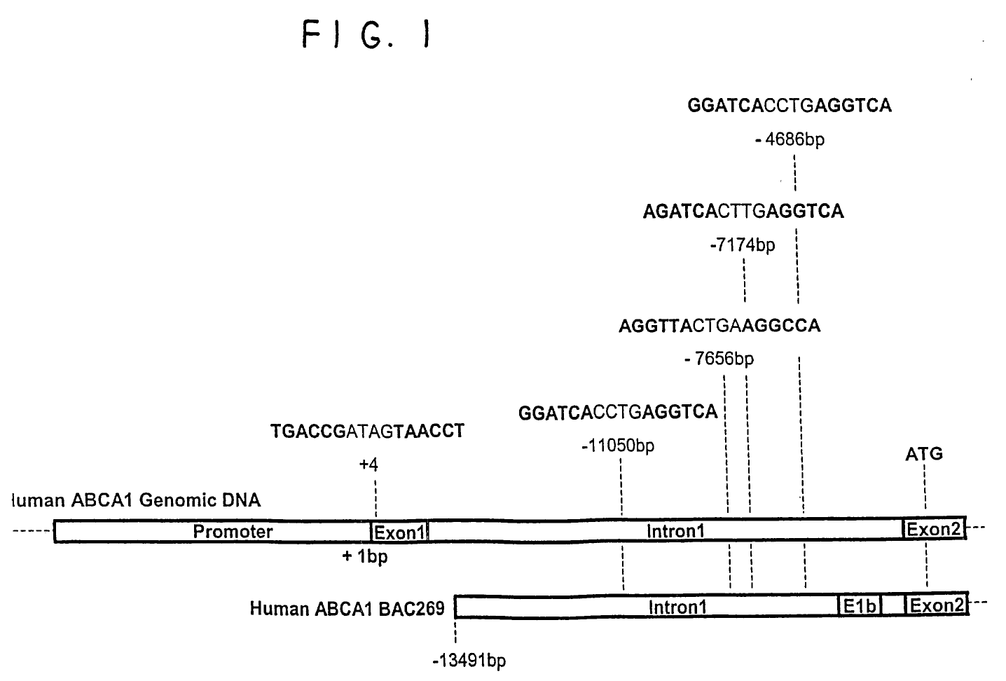

Cells were transfected for 3 hours by lipofection using ExGen 500 (Euromedex) in OPTIMEM 1. Medium was then replaced by Dulbecco's Modified Eagle's Medium (DMEM) containing 0.2% fetal calf serum and cells were incubated for 48 hours. Cell extracts were prepared and assayed for luciferase activity as described(15). Twenty-four hours before transfection, HepG2 cells, HUH7 cells, CaCo2 cells and RK13 cells were plated in 24-well plates in DMEM supplemented with 10% fetal calf serum at 5×10⁴ cells/well. Transfection mixes contained 100 ng of tkpGI3 reporter vector or pGI3 containing an 8 kb fragment from ABC1 intron I (pGI3-8 kb) (FIG. 1). Cos-1 cells were plated in 24-well plates in DMEM supplemented with 10% fetal calf serum at 5×10⁴ cells/well. Transfection mixes contained 50 ng of reporter plasmid (tkpGI3) containing multiple copies of the putative LXREs and 25 ng of LXRα and RXR expression plasmids, in the presence of the internal control β-gal expression vector. After transfection, cells were treated for 48 hours with 1 μM 22(R)-hydroxycholesterol (Sigma).

### Gel Mobility Shift Assay

LXRα and RXR were transcribed and translated in vitro using pCDNA3-LXRα and pSG5-mRXRα as templates and the TNT coupled transcription/translation system (Promega). Gel mobility shift assays (20 μl) contained 10 mM Tris (pH 8), 40 mM KCl, 0.1% Nonidet P-40, 6% glycerol, 1 mM dithiothreitol, 0.2 μg of poly (dldC), 1 μg herring sperm DNA, 2.5 μl each of in vitro synthesized LXRα and RXR proteins. The total amount of reticulocyte lysate was maintained constant in each reaction (5 μl) through the addition of unprogrammed lysate. After a 10 minute incubation on ice, 1 ng of ³²P-labeled oligonucleotide was added, and the incubation continued for an additional 10 minutes. DNA-protein complexes were resolved on a 6% polyacrylamide gel in 0.5×TBE. Gels were dried and subjected to autoradiography at ˜80° C.

### Multicopy Cloning

250 picomoles of each oligonucleotide to which half sites for BamH I and Bgl II restriction enzymes were added, were phosphorylated using PNK kinase (Roche), incubated for 5 minutes at 95° C., then 10 min at 65° C. and cooled to room temperature. Multimeric copies were then generated using T4 ligase, cloned in TKpGI3 vector and verified by sequencing.

### Generation of BAC Transgenic Mice

BAC's containing the ABC1 gene were identified by screening high density BAC grid filters from the RPCI-11 human male BAC library. The library was made at the Roswell Park Cancer Institute and was screened using 10× coverage gridded BAC filters purchased through Research Genetics. Four BACs containing ABC1 were sequenced as previously described(1,6). Version 1.7 of Clustal W with modifications was used for multiple sequence alignments with Boxshade for graphical enhancement. The 5′ end of BAC 269 is at position—13491 in intron 1 (ie. 13491 nucleotides from the 5′ end of exon 2). The address of BAC 269 from the RPCI-1 1 human male BAC library is 418 (number 4, letter I, number 8). This was chosen for further purification as it alone contained intron 1 sequence without the human ABC1 promoter, allowing us to test for functionality of these regulatory elements. The BAC's were purified for injection using the Qiagen Maxi Prep kit, followed by cesium chloride purification (16) and dialysis overnight. BAC's were quantified using agarose gel electrophoresis, and sets of 300 C57BL6/CBA eggs were injected with 30 ng of the purified BAC DNA. Founders were genotyped on DNA extracted from tail pieces, followed by subsequent PCR amplification of exon 2, exon 26, and exon 49 of the ABC1 gene. Positive founders were backcrossed to mice of the CBA background strain.

### Feeding of High Cholesterol Diets

BAC mice and control littermates were fed high fat/high cholesterol or a control chow diet for 7 days. The diets and water were provided ad libitum. Diets were purchased from Harlan Teklad with the high fat/high cholesterol diet (TD 90221) containing 15.75% fat, 1.25% cholesterol, and 0.5% sodium cholate. This diet has previously been shown to result in upregulation of ABC1 mRNA levels in mouse liver, assessed at 7 days after feeding (17). The control diet containing 0.5% sodium cholate (TD 99057).

### Detection of Alternate Transcripts Involving Intron 1 Sequence

In order to identify the transcript generated in the BAC mice lacking exon 1, ABC1 intron 1 sequence was searched by proscan for putative transcription sites, and several likely sites determined. Primers were synthesized, and Clontech marathon ready mouse and human liver cDNA was used to amplify putative transcripts using the predicted transcript primers and an ABC1 exon 3 reverse primer, following manufacturers instructions. Positive transcripts were confirmed using nested PCR, and sequenced. RNA was isolated from BAC transgenic and control littermate tissue using Trizol (Gibco BRL), and 5′ RACE was performed using previously described primers (18) following manufacturers instructions (Gibco BRL). All products were TA cloned (Invitrogen) and sequenced using an ABI 3100 automated DNA sequencer.

### Western Blot Analysis of the Distribution of ABC1

BAC transgenic mice and control liftermates were sacrificed by CO₂ inhalation, various tissues were isolated and placed in 500 μl of low salt lysis buffer containing complete protease inhibitor tablets (Boehringer Mannheim) on ice. The tissues were homogenized three times for 10 seconds each using a polytron homogenizer, and were sonicated for about 15 seconds. The resulting homogenate was spun down at 14000 rpm for 10 minutes at 4° C. in an eppendorf microcentrifuge, and the supernatant was aliquoted into tubes. Protein levels were quantified using the Lowry assay, and 100-150 μg of protein was separated on 7.5% polyacrylamide gels. The proteins were transferred to PVDF membranes (millipore) at 24 amps for 1 hour. Membranes were blocked in 10% skim milk in PBS for 1 hour at room temperature, followed by incubation in 4 μg/ml ABC1PEP4 protein polyclonal rabbit antibody directed against residues 2236 to 2259 in ABC1 in PBS/10% skim milk for 1 hour at room temperature (Wellington et al, manuscript in preparation). After several washes with PBS, 0.1% tween-20, a horse radish peroxidase conjugated secondary anti-rabbit antibody (BioRad) was added in 10% skim milk/PBS at a 1:4000 dilution followed by additional washes with PBS, 0.1% tween-20. The membrane was dipped in ECL (Amersham), and exposed to X-OMAT blue film (Kodak). Protein levels were quantitated using NIH image software.

### ABC1 Immunocytochemistry in Various Mouse Tissues

Protein expression in tissues does not necessarily give any indication of the cellular distribution of a protein and can lead to misinterpretation of the expression level of a protein in a particular cell. To further compare the in vivo cellular expression pattern of ABC1 protein in both ABC1/BAC transgenic mice and their wild-type littermates, immunocytochemistry was performed on a variety of fixed tissues using a novel rabbit polyclonal antibody raised against a specific ABC1 peptide (PEP4). Transgenic and wildtype mice were deeply anesthetized with pentobarbitol, injected intraperitoneally with 100 units of heparin in sterile water, and then transcardially pefused with cold 3% paraformaldehyde in 0.1M phosphate buffered saline (PBS). The brain, kidneys, and liver were removed from each mouse and post-fixed for 24-48 hours in the same fixative. For each organ 30-50 μm sections were cut on a vibrating microtome ( Vibratome). Sections were collected in sterile PBS at 4° C., rinsed in 0.1 M PBS with 0.3% Tween 20, and incubated in blocking solution (0.1% PBS with 0.3% Tween 20, 3% whole goat serum, and 5% bovine serum albumin) for 2 hours at room temperature.

Sections of liver and kidney were incubated for 48 hours with the ABC1 antibody. The brain of each mouse was processed for combined immunocytochemistry with a neuron-specific (NeuN, Chemicon) antibody, and the polyclonal rabbit antibody against ABC1 (PEP4). Sections were sequentially placed into primary antisera against ABC1 (diluted 1:2500 in block solution) and NeuN (dilution 1:50 in block solution) for 48 hours at 4° C. Following incubation with the primary antibody, sections were washed several times in blocking solution and incubated in secondary antibody for 48 hours at 4° C. Secondary antibodies (Molecular probes) were used as follows: goat anti-mouse Alexa 488 with NeuN at a dilution of 1:200, and goat anti-rabbit CY-3 with ABC1 primary at a dilution of 1:200.

Following further washes with 0.1 M PBS the sections were dry mounted on gelatin-coated slides, dehydrated by serial ethanol washes, and permanently mounted with Fluoromount (Gurr). Sections were analyzed using an upright fluorescence microscope (Zeiss), digital images captured on a CCD camera (Princeton Instrument Inc.). Combined and NeuN/ABC1 stained sections were processed into double immunofluorescence figures using Northern exposure image program. No staining was observed in control sections when primary antibody was omitted.

### Measurement of Plasma Lipid Levels and Apoprotein Levels

Mice were either bled by saphenous vein withdrawal or by cardiac puncture, and the collected blood added to tubes containing 5 μl of 0.1M EDTA. The tubes were spun in a microfuge at maximum speed for 5 minutes at 4° C., and the isolated plasma aliquoted and frozen. For the measurement of HDL-C, the plasma was mixed 1:1 with 20% PEG20, vortexed well, incubated at room temperature for 10 minutes, and spun at maximum speed for 5 minutes at room temperature(19). 20 μl of the resultant supernatant was added to 96 well maxisorp plates (Millipore), and 200 μl of Infinity cholesterol reagent (Sigma) was added to the wells. The plates were quantified in an ELISA reader at 492 nm. For the measurement of total cholesterol, 5 μl of plasma was added to the same plates, 200 μl of Infinity cholesterol reagent added, and the plate quantified as above. Triglycerides were measured by adding 10 μl of the plasma to a 96 well plate, followed by the addition of 100 μl of Solution1 from a triglyceride kit (Boehringer Mannheim). The samples were incubated at 4° C. for 10 minutes, followed by the addition of 100 μl of solution 2 from the kit. The plate was quantitated in an ELISA reader at 492 nm. FPLC (Pharmacia) separation of plasma lipoproteins was performed using two Superose™ 6 (Pharmacia) columns in series as previously described(19). Equal volumes of plasma (40 μl) from each mouse (n=8) in each group were pooled for the analysis. The cholesterol and TG content in each 0.5 ml fraction was assessed using commercially available enzymatic kits (Boehringer Mannheim). Apoproteins were measured as previously described (33,34).

### Establishment of Primary Fibroblast and Macrophage Cultures

For the isolation of macrophages, mice were injected intraperitoneally with 2 ml of 3% thioglycollate, and 3 days later were sacrificed by CO₂ inhalation. 5 ml of DMEM containing 10% FBS, L-glutamine, and penicillin/streptomycin (all Gibco BRL) was injected into the body cavity. The mouse was gently massaged, and the media was withdrawn and placed in tubes on ice, which were spun at 1200 rpm, for 5 minutes. The cell pellet was resuspended in 1 ml of the above media, cells were counted, and plated at a density of 5×10⁵ cells/ml in a volume of 300 μl. The cells were incubated in a humidified atmosphere of 5% CO₂, at 37° C. until used. Fibroblasts were isolated by dissecting the femurs of the mice, and triturating the above media through the bone to remove all bone marrow. The media was added to tubes on ice, spun at 1200 rpm, for 5 minutes at 4° C., and the pellet was resuspended in 10 ml of media. The cells were plated on 10 cm tissue culture plates (Corning) and left in a humidified atmosphere with 5% CO₂ at 4° C.

### Measurement of Efflux in Fibroblast and Macrophage Cells

Twenty-four hours post plating of the macrophage cells, [3H] cholesterol (2 μCi/ml) (NEN Dupont), and 50 μg/ml AcLDL (Intracel) were preincubated at 37° C. for 30 minutes. Media was made containing DMEM, 1% FBS, pen/strep, L-glutamine, 1 μM ACAT inhibitor (CI-976, a kind gift from Dr. Minghan Wang) and the preincubated 50 μg/ml AcLDL and [3H] cholesterol. The media in the 24 well plates were replaced with 300 μl of this cholesterol containing media, and the plates were incubated for 24 hours. The labeled media was then replaced with 300 μl of DMEM, pen/strep, L-glutamine, 1 μM ACAT inhibitor, and 0.2% defafted BSA (Sigma) or 10% delipidated serum (Sigma) for about 24 hours. The media was again replaced with 300 μl of DMEM, pen/strep, L-glutamine, either with or without 20 μg/ml ApoAl (Calbiochem), and the treatment compounds (9)cis-retinoic acid (Sigma) and 22(R)-hydroxy cholesterol (Steraloids). 24 hours later, the media was withdrawn and added to tubes and spun at maximum speed for 5 minutes at room temperature. 100 μl of the supernatant was added to scintillation vials, and radioactivity was quantitated. 200 μl of 0.1 N NaOH was added to each well containing the cells and incubated for 20 minutes at room temperature. 100 μl of this lysate was added to scintillation vials and quantified. Efflux was calculated as the total counts in the medium divided by the sum of the total count in the medium plus the cell lysate.

### EXAMPLE 2

### Intron 1 of ABC1 Contains Multiple Potential LXR Elements (LXREs)

In order to detect the presence of LXR elements, we scanned the human ABC1 intron1 region from position −1 to −24156 and discovered several putative regulatory elements. Among these were discovered several possible LXR elements containing imperfect direct repeats of the nuclear receptor half site AGGTCA separated by four nucleotides (DR4) (FIG. 1 and Table 1) (20).

LXR elements have been shown to be regulated by oxysterols(21,22) and are important transcription control points in cholesterol metabolism(23). An LXR element in exon1 of ABC1 had previously been shown to be active in the upregulation of the gene in vitro(10-12). We also scanned intron 1 of the mouse ABC1 gene to assess for sequence homology and LXREs to provide further evidence for the functional importance of these DNA elements. Similar LXREs in intron 1 of the human ABC1 gene were also seen in intron 1 of the mouse (Table 1, showing the position and sequence of LXR elements in the human ABC1 gene, with target sequence: AGGTCANNNAGGTCA—SEQ ID NO: 4).

TABLE 1

| LXRE | | % | | |
|---|---|---|---|---|
| Position | LXRE Sepuence* | Match | Ratio | Strand |
| Pro- | | | | |
| moter: | | | | |
| +1 | AGGTTA CTAT CGGTCA (5) | 83 | 10/12 | + |
| Intron | | | | |
| 1: | | | | |
| −11050 | GGATCA CCTG AGGTCA (6) | 83 | 10/12 | + |
| −7188 | AGATCA CTTG AGGTCA (7) | 92 | 11/12 | + |
| −7670 | AGGTTA CTGA AGGCCA (8) | 83 | 10/12 | − |
| −4696 | GGATCA CCTG AGGTCA (9) | 83 | 10/12 | − |

*The number in parentheses following each sequence is the SEQ ID NO.

### EXAMPLE 3

### Sequence in Intron 1 is Functional

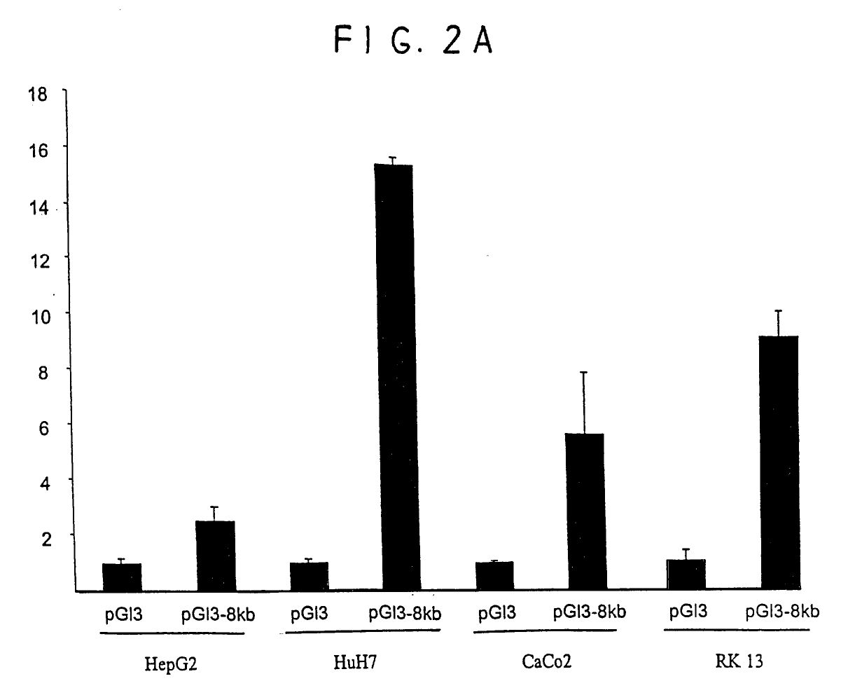

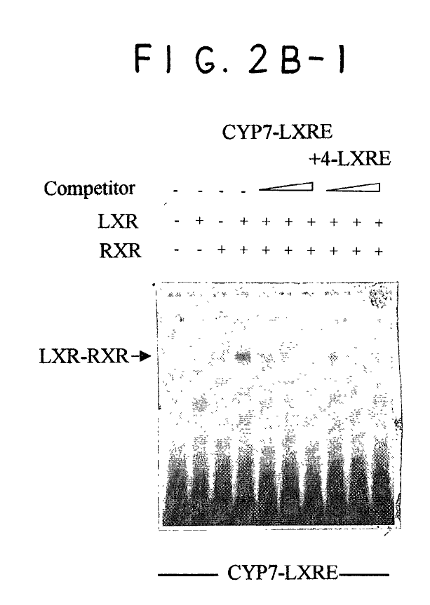

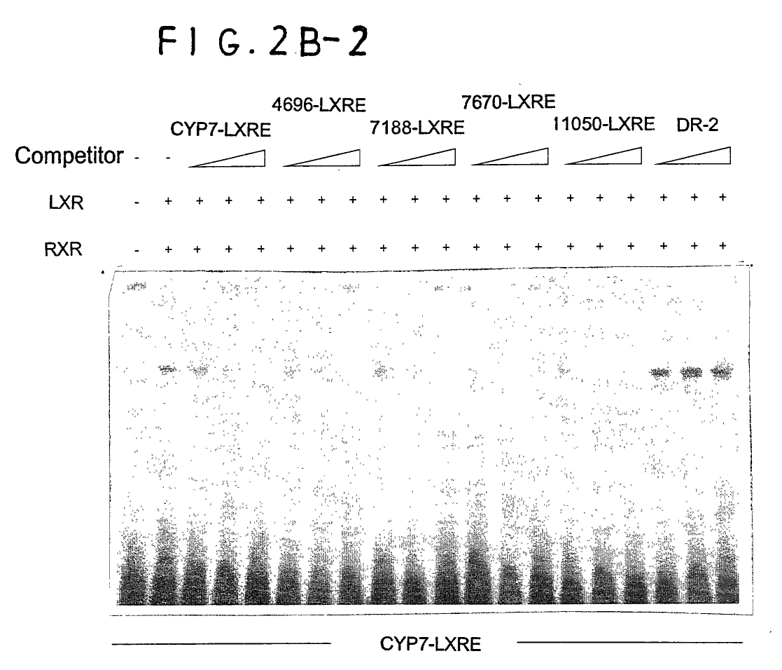

To investigate the function of potential LXREs in intron 1, we transfected an 8 kb fragment of intron 1 upstream of exon 2 spanning all potential LXREs into different cell types, including several hepatic (HepG2 and HuH7) and intestinal (CaCo₂) and renal (RK13) cell lines. Indeed, in these cell lines, a significant activation of the reporter gene was observed as compared to transfection of the empty pGI3 vector alone (FIG. 2A).

### EXAMPLE 4

### ABC1 Intron 1 Contains Functional LXRE's

To investigate whether these DR4 were indeed able to bind LXR-RXR heterodimers, gel retardation assays were performed (FIG. 2B). As shown before when LXRα and RXR proteins were incubated with the labeled CYP7-LXRE oligonucleotide in vitro, a complex was observed in the presence of the LXR-RXR heterodimer(22). An excess of unlabeled CYP7 competed efficiently for binding to the probe but no competition for binding was observed with a DR-2 oligonucleotide. As a control, the unlabeled LXRE previously described in ABC1 exon 1 (+4-LXRE) (10,11) also competed efficiently for binding. The signal was then competed with increasing quantities of each unlabeled putative LXRE. As shown in FIG. 2B, all four constructs competed for binding of the CYP7A probe in a dose dependant manner, although with seemingly different efficiencies.

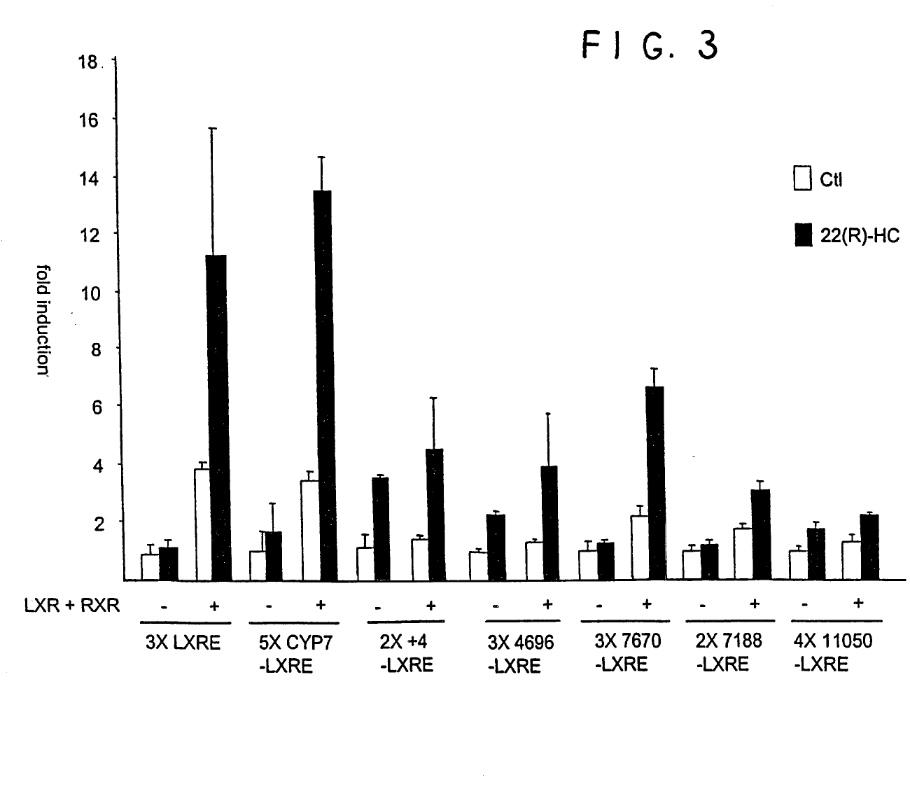

To determine whether these potential LXREs also possessed functional relevance, multiple copies of these oligonucleotides cloned in front of a luciferase reporter gene were assayed by cotransfection experiment with the expression plasmids for LXRα and RXR in cos-1 cells (FIG. 3). As previously described, 3 copies of the consensus LXRE (3×LXRE), 5 copies of the CYP7 LXRE (5×CYP7-LXRE), 2 copies of the +4 LXRE in ABC1 exon 1 (2×+4-LXRE) showed a strong activation in presence of cotransfected LXR and RXR plasmids (10,22). The 3 copies of the putative 4696 LXRE and 7670 LXRE of ABC1 intron 1 showed a 2-fold and 6-fold induction, respectively. In contrast, 2 copies of the 7188 LXRE and 4 copies of the 11050 LXRE showed a weaker activation by the LXR-RXR heterodimer.

### EXAMPLE 5

### Detection of Alternate Transcripts Involving Sequence in Intron 1

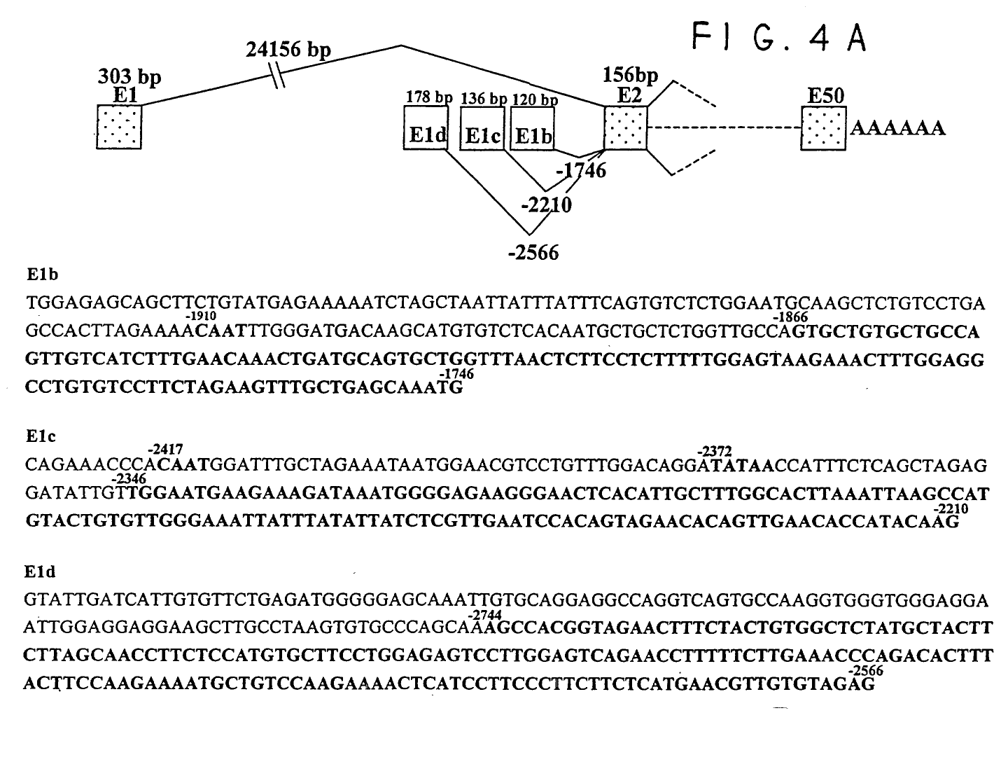

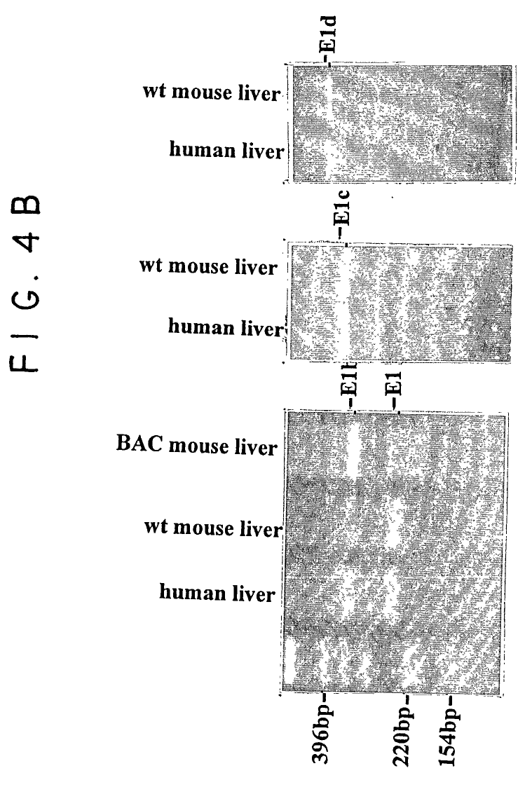

In order to elucidate the transcript generated by the BAC transgenic mice lacking exon 1 of the ABC1 gene, we performed RT-PCR and 5′ RACE. Utilizing these approaches followed by sequencing, we identified three novel transcripts containing published exon 2 and exon 3 sequences, and additional sequence from intron 1 (FIG. 4A). These transcripts were seen at different levels in hepatic tissue. For example, the transcript with exon 1 B was expressed at highest level in chow fed BAC transgenic compared to wild-type mice of the same diet. Furthermore, liver from wild-type mice had different levels of this transcript compared to humans (FIG. 4B).

### EXAMPLE 6

### ABC1 Protein is Increased in Humanized BAC Transgenic Mice

In order to determine if the LXRE's in intron 1 that we identified by bioinformatic and in vitro methods are functional in vivo, we generated mouse lines transgenic for the ABC1 gene where the promoter and exon 1 regions were deleted using BAC 269 to differentiate this from the prior known active LXR element (10) in the promoter. Protein expressed from this BAC would be predicted to be full-length with the translation initiation site in exon 2. We obtained several BAC founder lines, and all analyses were performed on two individual founder lines. These two founder lines had different copy numbers of human BACs. Southern blot analysis revealed founder XA with 3-4 BACs and founder XB with 1-2 BACs. This was paralleled by levels of protein with XA having 2× that is seen in human liver, compared to XB having below or close to endogeneous levels.

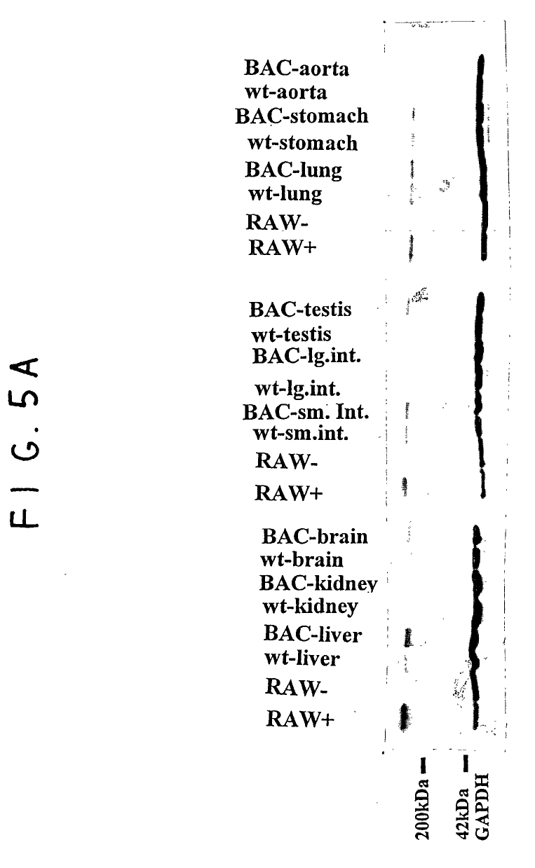

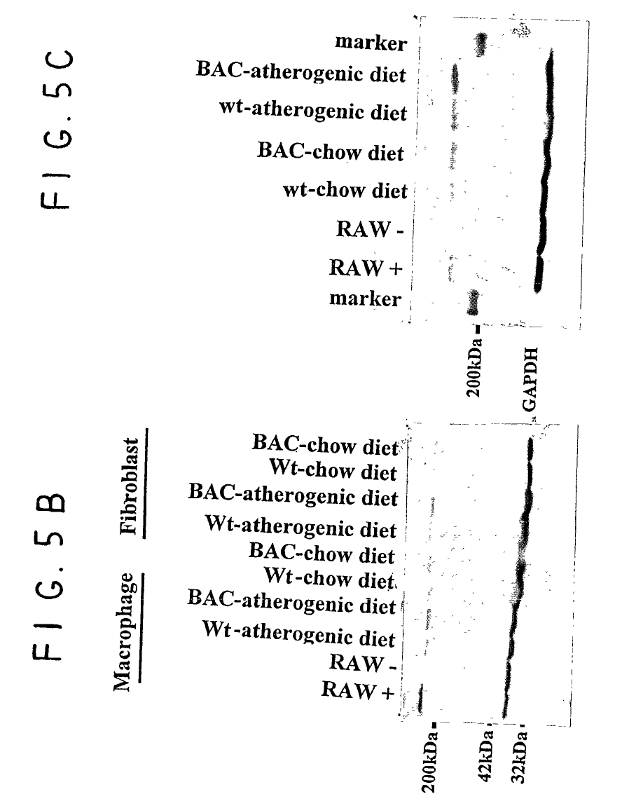

In order to determine if the ABC1 protein is expressed in the absence of its upstream promoter and exon 1 sequences, we first performed western blot analysis of several different tissues in the mice. We observed that there was indeed an increase in expression of ABC1 levels in the liver, small intestine, testis, stomach, and brain that was distinguishable by our anti ABC1 antibody (FIG. 5A) compared to nontransgenic mice. When the mice were fed an atherogenic diet, the levels of ABC1 protein were further induced in liver (FIG. 5B) and other tissues, giving us our first indication that ABC1 expression levels could be upregulated by elements separate from those found in its promoter and exon 1 regions and that LXRE's identified in intron I are indeed functional in vivo. This alternative promoter is also active in macrophages and fibroblasts as determined by the increase in ABC1 protein in these tissues in BAC transgenic mice and their response to oxysterol stimulation (FIG. 5C).

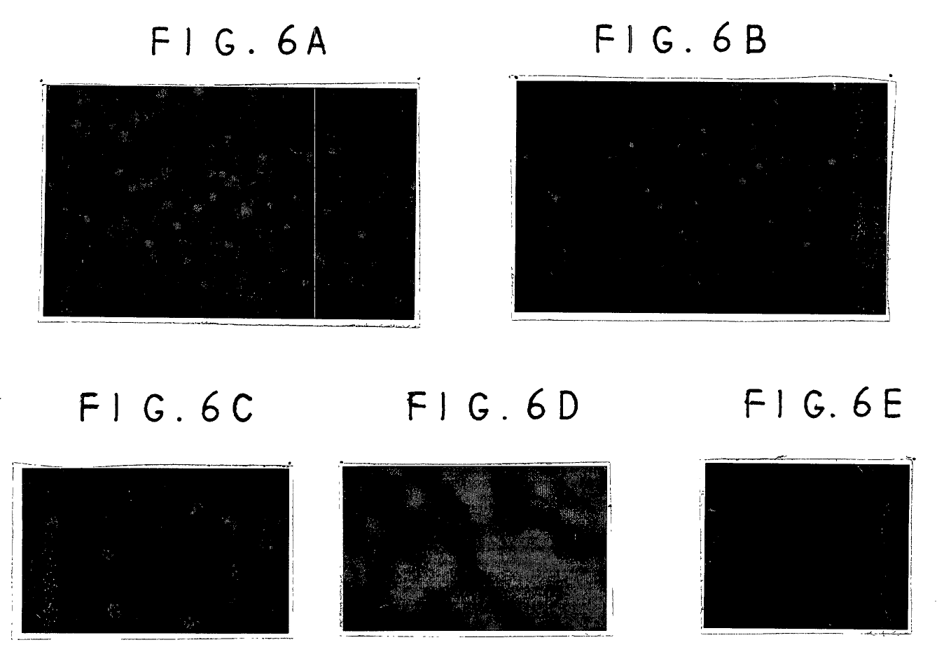

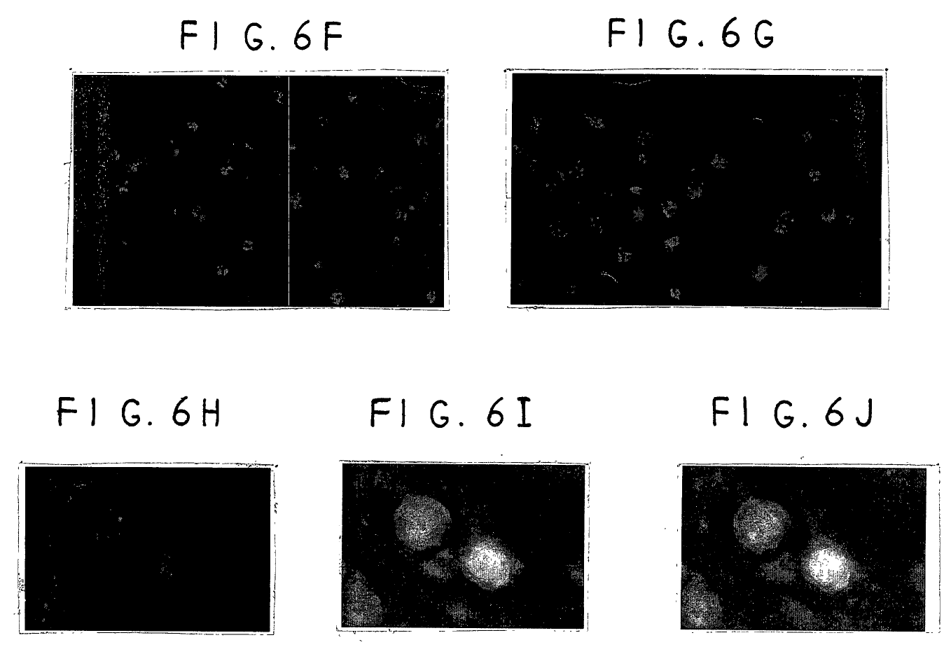

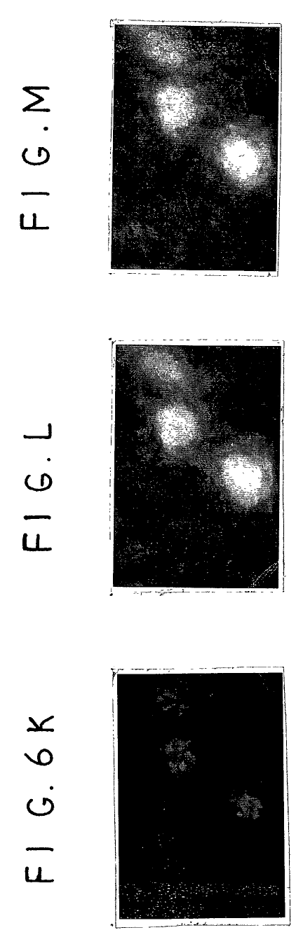

Immunohistochemical analysis on liver and brain (FIG. 6I) of wildtype liftermates and BAC transgenic mice showed qualitative increases in ABC1 expression in hepatocytes in the transgenic mice (FIGS. 6A-D). There was no alteration in the subcellular distribution of ABC1. For example, in the cortex, ABC1 is predominantly located in the nucleus of neurons in both transgenic and wildtype mice (FIGS. 6F-L).

### EXAMPLE 7

### ABC1 BAC Transgenic Mice Show Increased HDL-C Levels and Changes in Apoproteins

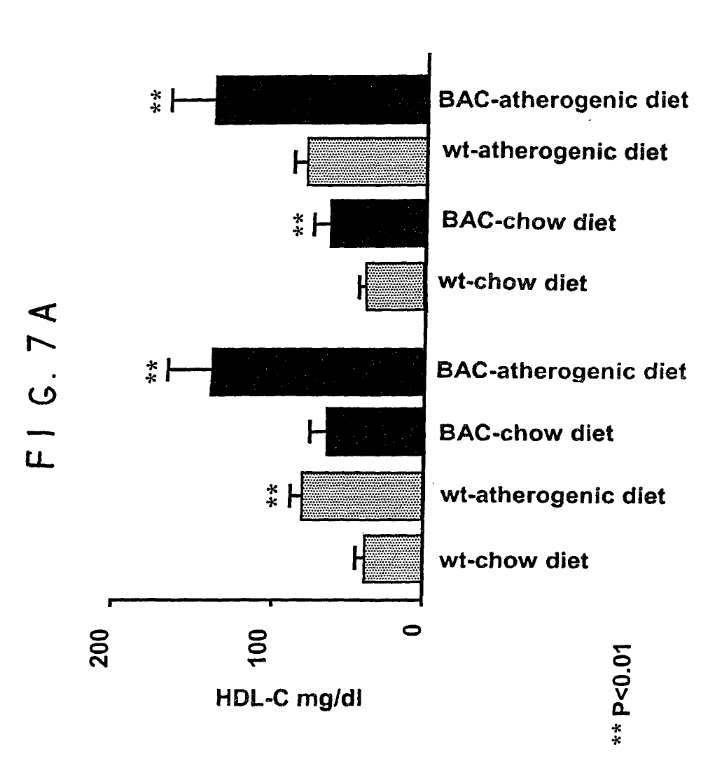

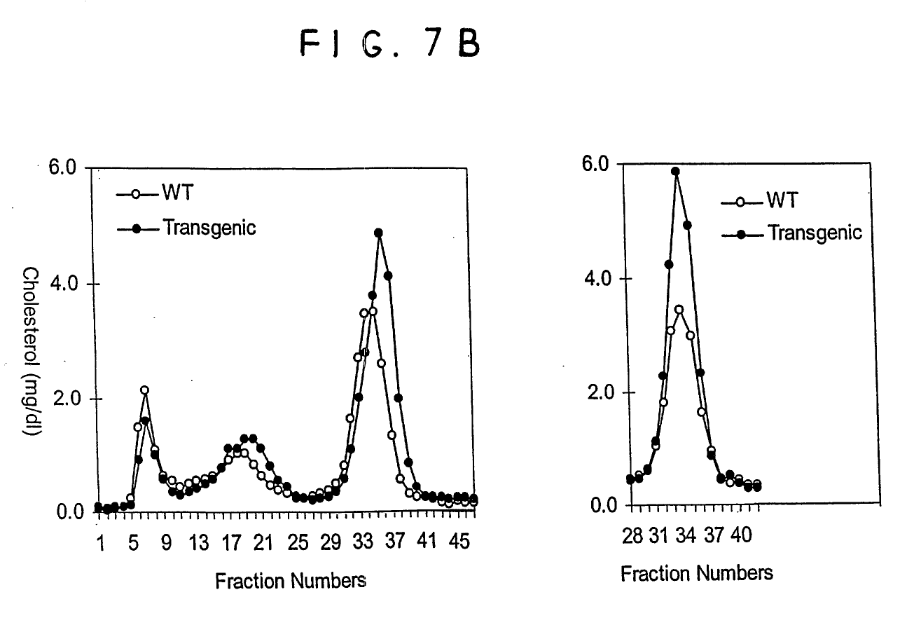

It was determined that the increase in ABC1 protein in the BAC mice resulted in an increase in its activity by measuring the plasma lipid levels in these mice. A significant increase in HDL-C levels in the ABC1 BAC transgenic mice when compared to control littermates, was seen both on a chow and atherogenic diet (n=x) (p=0.004 and 0.006 respectively) (FIG. 7A and Table 2 (the latter lists the analysis of plasma lipid profiles in ABC1 BAC transgenic mice). These data show that the alternate promoter in intron 1 is important and sufficiently functional to result in increased expression of ABC1 protein and resultant increase in HDL-C levels. Furthermore, in both the BAC transgenic mice and wildtype littermates the HDL levels increased significantly with a cholesterol-rich diet (p<0.001 and p=0.002, respectively, Table 2). Apoproteins Al and All were significantly increased in the BAC transgenic compared to wild-type mice on a chow diet (Table 4 lists the apoproprotein levels in BAC transgenic mice versus wild type (Wt)). To assess for qualitative differences in lipoprotein particles between the hABC1 BAC transgenics and their littermate controls, FPLC analysis was performed (FIG. 7B). HDL-C levels, as indicated by the total area of the HDL peak (fractions 30-38), were increased in the transgenic mice, compared to the non-transgenic controls. The size distribution of the HDL particles appears slightly different, as the peak appears in fraction 34 in the wildtype and 35 in transgenic mice. Remnant lipoproteins and LDL-C (fractions 12-20, 24-28, respectively) were not readily different between transgenics and controls. HDL-C levels were further increased on feeding with the atherogenic diet (FIG. 7B) with peaks occurring in the same fraction. Thus, increased HDL-C concentration reflects an increased number of HDL particles.

TABLE 2
Analysis of plasma lipid profiles in ABCA1 BAC transgenic mice

| | Wt | BAC ather- | Wt | BAC | Wt chow | Wt chow | BAC chow | Wt atherogenic |
|---|---|---|---|---|---|---|---|---|
| | chow diet | chow diet | ogenic diet | atherogenic diet | Vs. | Vs. Wt | Vs. BAC | Vs. |
| | n = 4 | n = 4 | n = 4 | n = 4 | BAC chow | atherogenic | atherogenic | BAC atherogenic |
| All mg/dl | Mean ± SD | Mean ± SD | Mean ± SD | Mean ± SD | P value | P value | P value | P value |
| Total | 65.29 ± 5.78 | 92.65 ± | 112.84 ± 14.2 | 180.68 ± 15.46 | 0.0003 | 0.01 | <0.0001 | 0.0007 |
| cholesterol | | 4.59 | | | | | | |
| HDL-C | 36.98 ± 1.76 | 61.18 ± | 77.84 ± 8.80 | 137.30 ± 27.91 | 0.0005 | <0.0001 | 0.002 | 0.007 |
| | | 11.02 | | | | | | |
| Triglycerides | 42.66 ± 7.55 | 49.95 ± | 50.60 ± 2.33 | 57.30 ± 4.43 | 0.17 | 0.09 | 0.04 | 0.04 |
| | | 3.62 | | | | | | |
| Non-HDL | 28.31 ± 3.77 | 31.47 ± | 35.00 ± 4.50 | 43.38 ± 4.68 | 0.34 | 0.07 | 0.028 | 0.04 |
| cholesterol | | 4.80 | | | | | | |

TABLE 3
Analysis of [3H]-cholesterol efflux to ApoAI in ABCA1 BAC transgenic mice

| | | | Wt | BAC | | | | |
|---|---|---|---|---|---|---|---|---|
| | Wt | BAC | atherogenic | atherogenic | Wt chow | Wt chow | BAC chow | Wt atherogenic |
| | chow diet | chow diet | diet | diet | Vs. | Vs. | Vs. | Vs. |
| | n = 4 | n = 4 | n = 4 | n = 4 | BAC chow | Wt atherogenic | BAC atherogenic | BAC atherogenic |
| | Mean ± SD | Mean ± SD | Mean ± SD | Mean ± SD | P value | P value | P value | P value |
| Macrophage | 0.31 ± 0.014 | 0.45 ± 0.0033 | 0.40 ± 0.022 | 0.54 ± 0.0077 | <0.0001 | 0.0004 | <0.0001 | <0.0001 |
| Macrophage | 0.46 ± 0.0085 | 0.49 ± 0.0067 | 0.52 ± 0.0054 | 0.59 ± 0.015 | 0.002 | <0.0001 | <0.0001 | 0.0002 |
| 22(R)-OH | | | | | | | | |
| chol. 9 | | | | | | | | |
| (cis) RA | | | | | | | | |
| Fibroblast | 0.12 ± 0.011 | 0.14 ± 0.017 | 0.16 ± 0.0014 | 0.29 ± 0.011 | 0.03 | 0.002 | <0.0001 | <0.0001 |
| Fibroblast | 0.19 ± 0.014 | 0.22 ± 0.015 | 0.27 ± 0.014 | 0.32 ± 0.0033 | 0.01 | 0.0002 | <0.0001 | 0.0003 |
| 22(R)-OH | | | | | | | | |
| chol. 9 | | | | | | | | |
| (cis) RA | | | | | | | | |

TABLE 4
Quantitation of Apoprotein levels in BAC transgenic mice*

| | | | WT (chow |
|---|---|---|---|
| | Wt | BAC | diet) vs. |
| | chow diet | chow diet | BAC (chow diet) |
| ApoAI | 0.43 ± 0.018 | 0.49 ± 0.018 | <0.05 |
| ApoAII | 0.35 ± 0.022 | 0.57 ± 0.032 | <0.0001 |
| ApoB | 0.15 ± 0.009 | 0.13 ± 0.0059 | <0.19 |
| ApoCIII | 0.39 ± 0.027 | 0.47 ± 0.032 | <0.07 |

*For wild type and BAC animals, the number in each group was 16 (n = 16) and the values are reported as the mean ± SD (standard deviation). Column 3 shows the P values.

### EXAMPLE 8

### ABC1 BAC Transgenic Mice Show Increased Efflux

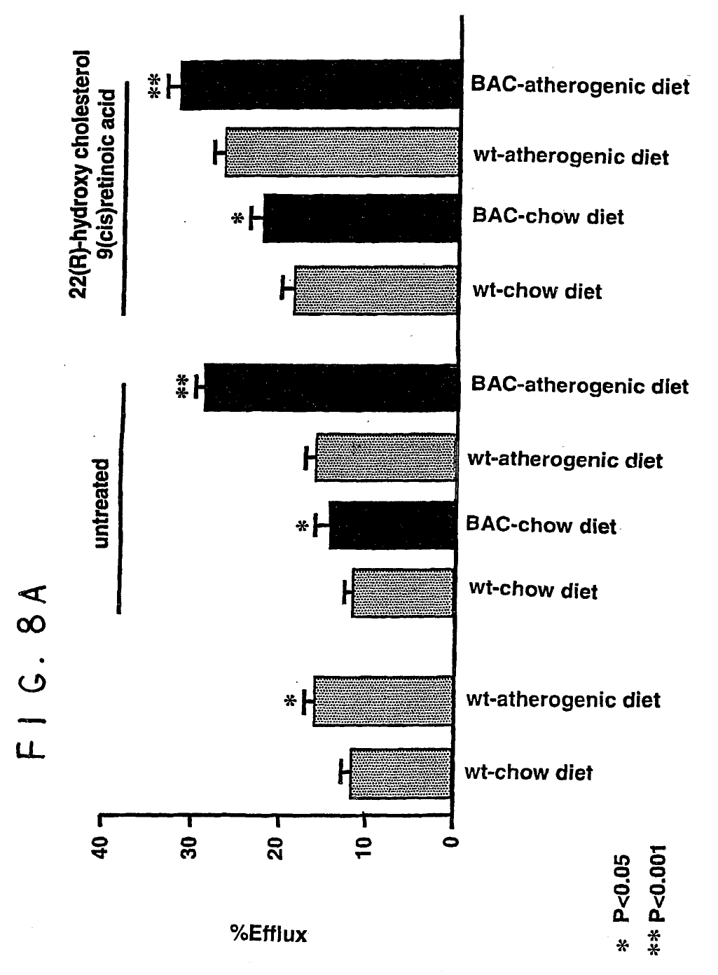

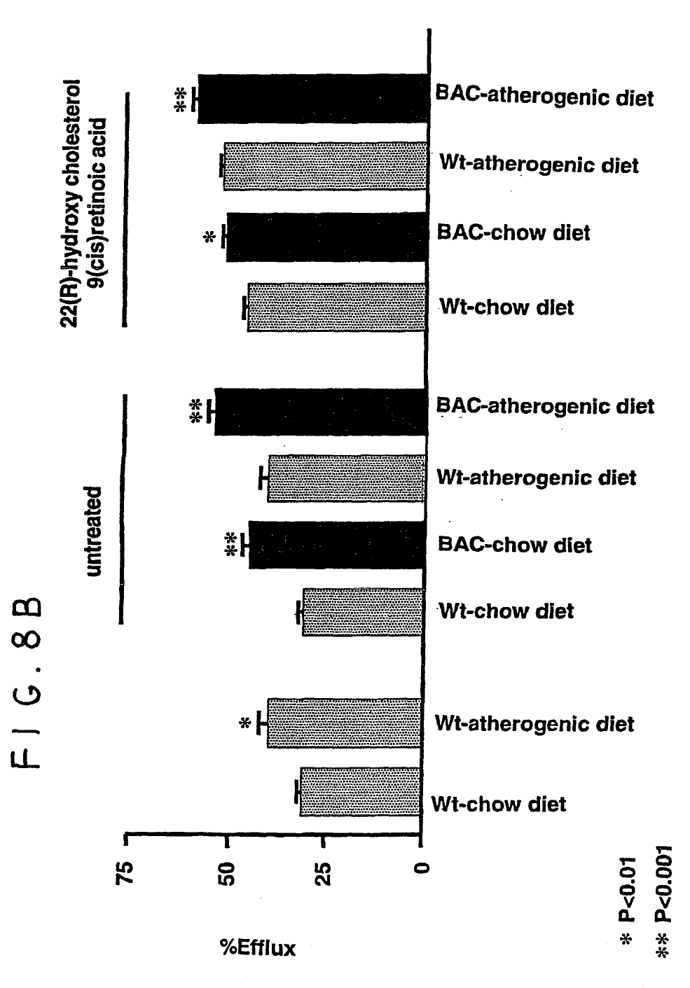

A defect in cholesterol and phospholipid removal mediated by apolipoproteins has been previously observed in ABCA1 defective Tangier disease fibroblasts(24) and ABCA1 has been shown to mediate cholesterol efflux to ApoAl or HDL from cells(4,25). In order to determine if there was an increase in efflux of cholesterol in the mice expressing high levels of ABCA1, we established primary peritoneal macrophage and fibroblast cultures from these mice. We observed increased efflux of [³H]-cholesterol to ApoAl from both peritoneal macrophage (FIG. 8A) and fibroblast (FIG. 8B and Table 3) cultures obtained from the transgenic mice when compared to wild-type littermates. These efflux levels were further significantly increased when the mice were fed the atherogenic diet (FIGS. 8A and 8B). We observed that there was no increase in efflux when ApoAl was omitted as the efflux acceptor. In addition, we observed that stimulation of cultures with 9(cis)-retinoic acid and 22(R)-hydroxy cholesterol also significantly upregulated the efflux levels from both the macrophage (FIG. 8A) and fibroblast cells (FIG. 8B and Table 3).

### EXAMPLE 9

### Methods of Identification of Single Nucleotide Polymorphisms

This invention additionally establishes that identification of single nucleotide polymorphisms (SNPs) and other polymorphisms and mutations in the ABC1 intragenic regions are useful for diagnostic and pharmacogenomic applications. In particular, identification of SNPs, polymorphisms, and mutations in intron 1 of ABC1 will be useful for predictive determination of whether one or more of the variant transcripts of the invention are increased or decreased in abundance in an individual or in any particular tissue of an individual. The relative abundance of the variant transcripts of the invention may be used to predict responsiveness to therapeutic agents, particularly agents which may modulate transcription of ABC1 (i.e. LXR/RXR agonists or antagonists) or agents which modulate ABC1 mRNA stability. Alternative diagnostic and pharmacogenomic applications of SNPs, polymorphisms, and mutations in ABC1 intragenic regions will be apparent to those skilled in the art.

All means of identifying DNA sequences specific to an individual are contemplated by this invention. One could easily ascertain whether these polymorphisms are present in a patient prior to the establishment of a drug treatment regimen for a patient having low HDL, a higher than normal triglyceride level, cardiovascular disease, or any other ABC1-mediated condition. It is possible that some these polymorphisms are, in fact, weak mutations. Individuals harboring such mutations may have an increased risk for cardiovascular disease; thus, these polymorphisms may also be useful in diagnostic assays. In general, the detection of single nucleotide polymorphism and single base mutation or variation requires a discrimination technique, optionally an amplification reaction and optionally a signal generation system. There are numerous techniques available for typing SNPs and allelic variations (for review, see Eberle & Kruglyak Genet Epidemiol 2000;19 Suppl 1:S29-35; Kennedy EXS 2000;89:1-10; Kao et al. Ann Acad Med Singapore 2000 May;29(3):376-82; Kao et al. Ann Acad Med Singapore 2000 May;29(3):376-82; Landegren et al., Genome Research, Vol. 8, pp. 769-776,1998; Nollau et al, Clin. Chem. 43,1114-1120, 1997 and in standard textbooks, for example ‘Laboratory Protocols for Mutation Detection’, Ed, Landegren, Oxford University Press, 1996 and ‘PCR’ 2nd Edition by Newton and Graham, BIOS Scientific Publishers limited,1997).

Techniques include direct sequencing (Carothers et al., BioTechniques, Vol. 7, pp. 494499,1989), single-strand conformation polymorphism (SSCP, Orita et al., Proc. Natl. Acad. Sci. USA, Vol. 86, pp. 2766-2770,1989), allele-specific amplification (Newton et al., Nucleic Acids Research, Vol. 17, pp. 2503-2516,1989), restriction digestion (Day and Humphries, Analytical Biochemistry, Vol. 222, pp. 389395,1994), restriction fragment length polymorphism (RFLP) and hybridization assays. Other methods include high density arrays, mass spectrometry, molecular beacons, peptide nucleic acids, and the mismatch cleavage based assays. These include but are not limited to bacteriophage T4 endonuclease VII (U.S. Pat. No. 6,110,684 issued Aug. 29, 2000; U.S. Pat. No. 6,183,958 issued Feb. 06, 2001, U.S. Pat. No.5,958,692, U.S. Pat. No. 5,851,770, WO 00/18967 Apr. 06, 2000; WO 00/50639 Aug. 31, 2000) WO 00/18967). 5′ nucleases and/or 3′ exonucleases (U.S. Pat. No. 5,888,780, WO 98/50403A1, U.S. Pat. No. 5,719,028, WO 00/66607; WO056925) and others such as WO 073766, WO050871, WO 00/66607).

Techniques can also be classified as either target amplification or signal amplification. Target amplification involves the amplification (i.e., replication) of the target sequence to be detected, resulting in a significant increase in the number of target molecules. Target amplification strategies include the polymerase chain reaction (PCR), strand displacement amplification (SDA), and nucleic acid sequence based amplification (NASBA). Signal amplification strategies include the ligase chain reaction (LCR), cycling probe technology (CPT), invasive cleavage techniques such as Invader™ technology, Q-Beta replicase (QBR) technology, and the use of “amplification probes” such as “branched DNA” that result in multiple label probes binding to a single target sequence.

Further assays include, but are not limited to, ligation based assays, cleavage based assays (mismatch and invasive cleavage such as Invader™), single base extension methods (see WO 92/15712, EP 0 371 437 B l, EP 0317 074 B l; Pastinen et al., Genome Res. 7:606-614 (1997); Syvänen, Clinica Chimica Acta 226: 225-236 (1994); and WO 91/13075).

The polymerase chain reaction (PCR) is widely used and described, and involves the use of primer extension combined with thermal cycling to amplify a target sequence; see U.S. Pat. Nos. 4,683,195 and 4,683,202, and PCR Essential Data, J. W. Wiley & sons, Ed. C. R. Newton, 1995, all of which are incorporated by reference. In addition, there are a number of variations of PCR which also find use in the invention, including “quantitative competitive PCR” or “QC-PCR”, “arbitrarily primed PCR” or “AP-PCR”, “immuno-PCR”, “Alu-PCR”, “PCR single strand conformational polymorphism” or “PCR-SSCP”, allelic PCR (see Newton et al. Nucl. Acid Res. 17: 2503 91989); “reverse transcriptase PCR” or “RT-PCR”, “biotin capture PCR”, “vectorette PCR”. “panhandle PCR”, and “PCR select cDNA subtraction”, Multiplex PCR amplification of SNP loci with subsequent hybridization to oligonucleotide arrays has been shown to be an accurate and reliable method of simultaneously genotyping at least hundreds of SNPs; see Wang et al., Science, 280: 1077 (1998); Schafer et al., Nature Biotechnology 16: 33-39 (1998).

Strand displacement amplification (SDA) is generally described in Walker et al., in Molecular Methods for Virus Detection, Academic Press, Inc., 1995, and U.S. Pat. Nos. 5,455,166 and 5,130,238, all of which are hereby incorporated by reference. Nucleic acid sequence based amplification (NASBA) is generally described in U.S. Pat. No. 5,409,818 and “Profiting from Gene-based Diagnostics”, CTB International Publishing Inc., N. J., 1996, both of which are incorporated by reference in their entirety.

Cycling probe technology (CPT) is a nucleic acid detection system based on signal or probe amplification rather than target amplification, such as is done in polymerase chain reactions. Cycling probe technology relies on a molar excess of labeled probe that contains a scissile linkage of RNA. Upon hybridization of the probe to the target, the resulting hybrid contains a portion of RNA: DNA. This area of RNA: DNA duplex is recognized by RNAse H and the RNA is excised, resulting in cleavage of the probe. The probe now consists of two smaller sequences which may be released, thus leaving the target intact for repeated rounds of the reaction. The unreacted probe is removed and the label is then detected. CPT is generally described in U.S. Pat. Nos. 5,011,769,5,403,711, 5,660,988, and 4,876,187, and PCT published applications WO 95/05480, WO 95/1416, and WO 95/00667, all of which are specifically incorporated herein by reference. Invader™ technology is based on structure-specific polymerases that cleave nucleic acids in a site specific manner. Two probes are used: an “invader” probe and a “signaling” probe, that adjacent hybridize to a target sequence with a non-complementary overlap. The enzyme cleaves at the overlap due to its recognition of the “tail”, and releases the “tail” with a label. This can then be detected. The Invader technology is described in U.S. Pat. Nos. 5,846,717; 5,614,402; 5,719,028; 5,541,311; and 5,843,669, all of which are hereby incorporated by reference.

The oligonucleotide ligation assay (OLA), sometimes referred to as the ligation chain reaction (LCR)), involve the ligation of at least two smaller probes into a single long probe, using the target sequence as the template for the ligase. See generally U.S. Pat. Nos. 5,185,243, 5,679,524 and 5,573,907; EP 0 320 308 B1; EP 0 336 731 B1; EP 0 439 182 B1; WO 90/01069; WO 89/12696; and WO 89/09835.

“Rolling circle amplification” is based on extension of a circular probe that has hybridized to a target sequence. A polymerase is added that extends the probe sequence. As the circular probe has no terminus, the polymerase repeatedly extends the circular probe resulting in concatamers of the circular probe. As such, the probe is amplified. Rolling-circle amplification is generally described in Baner et al. (1998) Nuc. Acids Res. 26: 5073-5078; Barany, F. (1991) Proc. Natl. Acad. Sci. USA 88: 189-193; and Lizardi et al. (1998) Nat Genet. 19: 225-232, all of which are incorporated by reference in their entirety.

Branched DNA signal amplification (BDNA) relies on the synthesis of branched nucleic acids, containing a multiplicity of nucleic acid “arms” that function to increase the amount of label that can be put onto one probe. This technology is generally described in U.S. Pat. Nos. 5,681,702, 5,597,909 ,5,545,730, 635,352,5,594,118, 5,359,100, 5,124,246 and 5,681,697, all of which are hereby incorporated by reference. Similarly, dendrimers of nucleic acids serve to vastly increase the amount of label that can be added to a single molecule, using a similar idea but different compositions. This technology is as described in U.S. Pat. No. 5,175,270 and Nilsen et al., J. Theor. Biol. 187: 273 (1997), both of which are incorporated herein by reference.

Other methods include mismatch detection techniques using enzymatic cleavage such as resolvase (Variagenics resolvase, is bacteriophage T4 endonuclease VII, U.S. Pat. No. 6,110,684 issued Aug. 29, 2000; U.S. Pat. No. 6,183,958 issued Feb. 06, 2001, U.S. Pat. No. 5,958,692, U.S. Pat. No. 5,851,770, WO 00/18967 Apr. 06, 2000; WO 00/50639 Aug. 31, 2000) WO 00/18967). The use of 5′ nucleases and/or 3′ exonucleases for target dependent reactions using cleavage structures (Third Wave U.S. Pat. No. 5,888,780, WO 98/50403A1, U.S. Pat. No. 5,719,028, Aclara (WO 00/66607; W0056925). Orchid Biosciences (WO 073766, W0050871, WO 00/66607).

### OTHER EMBODIMENTS

All publications, patents, and patent publications mentioned in this specification are herein incorporated by reference to the same extent as if each independent publication, patent, and patent publication was specifically and individually indicated to be incorporated by reference.

While the invention has been described in connection with specific embodiments thereof, it will be understood that it is capable of further modifications. This application is intended to cover any variations, uses, or adaptations following, in general, the principles of the invention and including such departures from the present disclosure within known or customary practice within the art to which the invention pertains and may be applied to the essential features set forth herein.

### REFERENCES

1. Brooks-Wilson, A. et al., (1999) Nat.Genet 22, 336-345

2. Bodzioch, M. et al., (1999) Nat. Genet. 22, 347-351

3. Rust, S. et al., (1999) Nat.Genet. 22, 352-355

4. Marcil, M., et al., (1999) Lancet354, 1341-1346

5. Lawn, R. M. et al., (1999) J.Clin.Invest. 104, R25-R31

6. Clee, S. M. et al., (2000) J.Clin. Invest. 106,1263-1270

7. Christiansen-Weber, T. et al., (2000) Am.J.Pathol. 157, 1017-1029

8. McNeish, J. et al., (2000) Proc.Natl.Acad.Sci 97, 4245-4250

9. Attie, A. (2000) Circulation

10. Schwartz, K. et al., (2000) Biochem.Biophys.Res.Commun. 274, 794-802

11. Costet, P. et al., (2000) J.Biol.Chem. 275, 28240-28245

12. Chawla, A. et al., (2001) Mol.Cell. 7, 161-171

13. Aebersold, R. et al., (2000) Annals of the New York Academy of Sciences 919, 33-47.

14. Gygi, S. P. et al., (1999) Mol.Cell.Biochem. 19,1720-1730

15. De lrive, P. et al., (2000) FEBS Lett. 471, 34-38

16. Maniatis. (2001)

17. Hedrick, C. C.et al., (2000) Arterioscler.Thromb.Vasc.Biol. 20, 1946-1952

18. Pullinger, C. R. et al., (2000) Biochem.Biophys.Res.Commun. 271, 451-455

19. Liu, M. -S. et al., (1994) J.Biol.Chem. 269,11417-11424

20. Willy, P. J. et al., (1995) Genes Dev. 9, 1033-1045

21. Janowski, B. A. et al., (1996) Nature 383, 728-731

22. Lehmann, J. M. et al., (1997) J.Biol.Chem. 272, 3137-3140

23. Peet, D. J. et al., (1998) Cell93,693-704

24. Marcil, M. et al., (1999) Arterioscler.Thromb.Vasc.Biol. 19,159-169

25. Langmann, T. et al., (1999) Biochem.Biophys.Res.Commun. 257, 29-33

26. Bruening, W. et al., (1992) Nat.Genet. 1, 144-148

27. Lopez, A. J. (1998) Annu.Rev.Genetics 32, 279-305

28. Adams, M. D. et al., (1996) Curr.Opin.Cell Biol. 8, 331-339

29. Boggs, R. T. et al., (1987) Cell 50, 739-747

30. Lejeune, F. et al., (2000) J.Biol.Chem. 276, 7850-7858

31. McQueen, K. L. et al., (2001) Immunogenetics 52, 212-223

32. Fatyol, K. et al., (1999) Mol.Gen.Genet. 261, 337-345

33. Rogler, G. et al., (1995) Arterioscler.Thromb.Vasc.Biol. 15, 683-690

34. Francis, G. A. et al., (1995) J.Clin.Invest. 96, 78-87

What is claimed is:

1. An isolated polynucleotide comprising:
(a) a polynucleotide that encodes a polypeptide having ATP-Binding Cassette transporter 1 (ABCA1) activity wherein said isolated polynucleotide further comprises a sequence selected from the group consisting of exons E1b, E1c and E1d and does not comprise Exon 1 of ABCA1, and (b) the full complement of (a).

2. The isolated polynucleotide of claim 1 wherein the polynucleotide of (a) that encodes a polypeptide is at least 95% identical to the sequence of SEQ ID NO: 1.

3. The isolated polynucleotide of claim 1 wherein the polynucleotide of (a) that encodes a polypeptide is identical to the sequence of SEQ ID NO: 1.

4. The isolated polynucleotide of claim 1 wherein the polynucleotide of (a) that encodes a polypeptide is at least 95% identical to the sequence of SEQ ID NO: 2.

5. The isolated polynucleotide of claim 1 wherein the polynucleotide of (a) that encodes a polypeptide is identical to the sequence of SEQ ID NO: 2.

6. The isolated polynucleotide of claim 1 wherein the polynucleotide of (a) that encodes a polypeptide is at least 95% identical to the sequence of SEQ ID NO: 3.

7. The isolated polynucleotide of claim 1 wherein the polynucleotide of (a) that encodes a polypeptide is identical to the sequence of SEQ ID NO: 3.

8. An isolated polynucleotide comprising a nucleotide sequence selected from the group consisting of SEQ ID NO: 1, 2 and 3 and wherein said isolated polynucleotide does not comprise Exon 1 of ABCA1.

9. The isolated polynucleotide of claim 1 wherein said polynucleotide of (a) is a cDNA.

10. A vector comprising a polynucleotide that encodes an active ABCA1 polypeptide and wherein said polynucleotide comprises a member selected from the group consisting of E1b, E1c and E1d but does not comprise Exon 1 of ABCA1.

11. The vector of claim 10 wherein said vector is a bacterial artificial chromosome (BAC).

12. A recombinant cell containing the vector of claim 10.

13. A recombinant cell containing the vector of claim 11.

14. The recombinant cell of claim 13 wherein said cell is a mammalian cell.

15. The recombinant cell of claim 14 wherein said mammalian cell is a human cell.

16. A non-human transgenic animal wherein the genome of one or more cells of said animal comprises a polynucleotide that encodes an active ABCA1 polypeptide and wherein said polynucleotide comprises a member selected from the group consisting of E1b, E1c and E1d but does not comprise Exon 1 of ABCA1.

17. The non-human transgenic animal of claim 16 wherein said animal has been produced using the vector of claim 10.

18. The non-human transgenic animal of claim 16 wherein said animal has been produced using the vector of claim 11.

19. The non-human transgenic animal of claim 16 wherein said animal is a mouse.

20. A method for identifying a compound that modulates ABCA1 biological activity comprising:
(a) contacting a compound with a polynucleotide that encodes a polypeptide having ABCA1 activity wherein said polynucleotide further comprises a sequence selected from the group consisting of exons E1b, E1c and E1d and does not comprise Exon 1 of ABCA1 and under conditions promoting said contacting and promoting expression of said polynucleotide, and
(b) determining a difference in expression of said polynucleotide in the presence of said compound compared to when said compound is not present, thereby identifying a compound that modulates ABCA1 biological activity.

21. The method of claim 20 wherein said difference in expression is an increase in expression.

22. The method of claim 20 wherein said expression is determined by determining the production of RNA encoded by said polynucleotide.

23. The method of claim 20 wherein said expression is determined by determining the production of polypeptide encoded by said polynucleotide.

24. The method of claim 20 wherein the polynucleotide that encodes a polypeptide having ABCA1 biological activity is a cDNA.

25. The method of claim 20 wherein said polynucleotide is present in a recombinant cell of claim 12.

26. The method of claim 20 wherein said polynucleotide is present in a recombinant cell of claim 13.

27. The method of claim 20 wherein said polynucleotide is present in a transgenic animal of claim 17.

28. The method of claim 20 wherein said polynucleotide is present in a transgenic animal of claim 18.

29. The method of claim 20 wherein said polynucleotide is present in a transgenic animal of claim 19.

30. A method for identifying a compound that modulates plasma lipid levels in an animal comprising administering to an animal an effective amount of a compound that modulates the expression of ABCA1 biological activity when such modulation is determined by the method of claim 20 and determining a change in plasma lipid levels as compared to when said compound has not been administered to said animal.

31. The method of claim 30 wherein said lipid is a triglyceride.

32. The method of claim 30 wherein said lipid is a phospholipid.

33. The method of claim 30 wherein said lipid is cholesterol.

34. The method of claim 33 wherein said cholesterol is part of HDL-cholesterol.

35. The method of claim 30 wherein said animal is a mammal.

36. The method of claim 35 wherein said mammal is a human being.

37. The method of claim 30 wherein said animal is a non-human transgenic animal.

38. The method of claim 30 wherein said non-human transgenic animal is the non-human transgenic animal of claim 18 or 19.

39. The method of claim 37, 38 or 39 wherein said animal is a mouse.

40. A method of lowering plasma lipid level in a mammal exhibiting elevated plasma lipid level comprising administering to said animal an effective amount of a compound first identified as lowering plasma lipid levels using the method of claim 20 or 30.

41. The method of claim 40 wherein said mammal is a human being.

42. The method of claim 40 wherein said lipid is a triglyceride.

43. The method of claim 40 wherein said lipid is a phospholipid.

44. The method of claim 40 wherein said lipid is cholesterol.

45. The method of claim 40 wherein said lipid is HDL-cholesterol.

46. A process for producing a product comprising identifying an agent according to the method of claim 20 or 30 wherein said product is the data collected with respect to said agent as a result of said process and wherein said data is sufficient to convey the chemical structure and/or properties of said agent.
